(19) United States
(12) Patent Application Publication
(10) Pub. No.: US 2025/0312110 A1
(43) Pub. Date: Oct. 9, 2025

(54) SURGICAL ROBOTICS SYSTEM WITH INTRAOPERATIVE HAPTICS GENERATION

(71) Applicant: MAKO Surgical Corp., Weston, FL (US)

(72) Inventor: David Bowling, Los Ranchos, NM (US)

(21) Appl. No.: 19/085,073

(22) Filed: Mar. 20, 2025

Related U.S. Application Data
(60) Provisional application No. 63/569,296, filed on Mar. 25, 2024.

(51) Int. Cl. A61B 34/30; A61B 17/00; A61B 17/16; A61B 34/10; A61B 34/20; A61B 90/00

(52) U.S. Cl. CPC A61B 34/30; A61B 17/16; A61B 34/10; A61B 90/36; A61B 2017/00477; A61B 2034/107; A61B 2034/2055; A61B 2090/365

(57) ABSTRACT

A surgical system comprising a robot, a cutting tool coupled to the robot, and a computing system. The computing system is programmed to capture a plurality of positions of the cutting tool in a coordinate frame as the cutting tool contacts a plurality of locations on a patient, generate, using the plurality of positions and a projection from the plurality of positions to a selected cut depth, a virtual boundary in the coordinate frame, and control the robot using the virtual boundary to guide the cutting tool in executing a resection in accordance with the virtual boundary.

### CROSS-REFERENCE TO RELATED PATENT APPLICATIONS

This application claims the benefit of and priority to U.S. Provisional Application No. 63/569,296, filed on Mar. 25, 2024, the entire disclosure of which is hereby incorporated by reference herein.

### BACKGROUND

The present disclosure relates generally to robotic assisted surgical systems. Robotic assistance can be provided to assist in execution of a surgery, for example, orthopedic and trauma surgeries, such as joint replacement surgeries. Improvements and additional features for robotically-assisted surgery can improve surgeon experience and patient outcomes.

### SUMMARY

At least one embodiment relates to a surgical system. The surgical system comprises a robot, a cutting tool coupled to the robot, and a computing system. The computing system is programmed to capture a plurality of positions of the cutting tool in a coordinate frame as the cutting tool contacts a plurality of locations on a patient. The computing system is further programmed to generate, using the plurality of positions and a projection from the plurality of positions to a selected cut depth, a virtual boundary in the coordinate frame, and control the robot using the virtual boundary to guide the cutting tool in executing a resection in accordance with the virtual boundary.

Another embodiment relates to a method of controlling a robotic device. The method comprises capturing a plurality of positions of a cutting tool of a robotic device in a coordinate frame as the cutting tool contacts a plurality of locations on a patient. The method further comprises generating, using the plurality of positions and a projection from the plurality of positions to a selected cut depth, a virtual boundary in the coordinate frame, and controlling the robotic device using the virtual boundary to guide the cutting tool in executing a resection in accordance with the virtual boundary.

Another embodiment relates to one or more non-transitory computer-readable media storing program instructions that, when executed by one or more processors, cause the one or more processors to perform operations. The operations comprise capturing a plurality of positions of a cutting tool of a robotic device in a coordinate frame as the cutting tool contacts a plurality of locations on a patient. The operations further comprise generating, using the plurality of positions and a projection from the plurality of positions to a selected cut depth, a virtual boundary in the coordinate frame, and controlling the robotic device using the virtual boundary to guide the cutting tool in executing a resection in accordance with the virtual boundary.

This summary is illustrative only and should not be regarded as limiting.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a perspective view of a surgical system, according to an exemplary embodiment.

FIG. 2 is a perspective view of a surgical tool of the surgical system of FIG. 1, according to an exemplary embodiment.

FIG. 3 is a block diagram of the surgical system of FIG. 1, according to an exemplary embodiment.

FIG. 4 is a storyboard-style illustration showing a process for establishing a coordinate frame using the surgical system of FIG. 1, according to an exemplary embodiment.

FIG. 5 is a storyboard-style illustration showing a process for establishing a coordinate frame using the surgical system of FIG. 1, according to an exemplary embodiment.

FIG. 6 is a storyboard-style illustration showing a process for registering an anatomy of a patient using the surgical system of FIG. 1, according to an exemplary embodiment.

FIG. 7 is a storyboard-style illustration showing a process for tracking an anatomy of a patient using the surgical system of FIG. 1, according to an exemplary embodiment.

FIG. 8 is a storyboard-style illustration showing a process for generating a virtual boundary using the surgical system of FIG. 1, according to an exemplary embodiment.

FIG. 9 is a storyboard-style illustration showing a process for generating a virtual boundary using the surgical system of FIG. 1, according to an exemplary embodiment.

FIG. 10 is a storyboard-style illustration showing a process for generating a virtual boundary using the surgical system of FIG. 1, according to an exemplary embodiment.

FIG. 11 is a storyboard-style illustration showing a process for generating a virtual boundary using the surgical system of FIG. 1, according to an exemplary embodiment.

FIG. 12A is a storyboard-style illustration showing a process for generating a virtual boundary using the surgical system of FIG. 1, according to an exemplary embodiment.

FIG. 12B is a storyboard-style illustration showing a process for generating a virtual boundary using the surgical system of FIG. 1, according to an exemplary embodiment.

FIG. 13 is a storyboard-style illustration showing a process for generating a virtual boundary using the surgical system of FIG. 1, according to an exemplary embodiment.

FIG. 14 is a flow diagram of a process of providing a virtual boundary that can be executed by the surgical system of FIG. 1, according to an exemplary embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

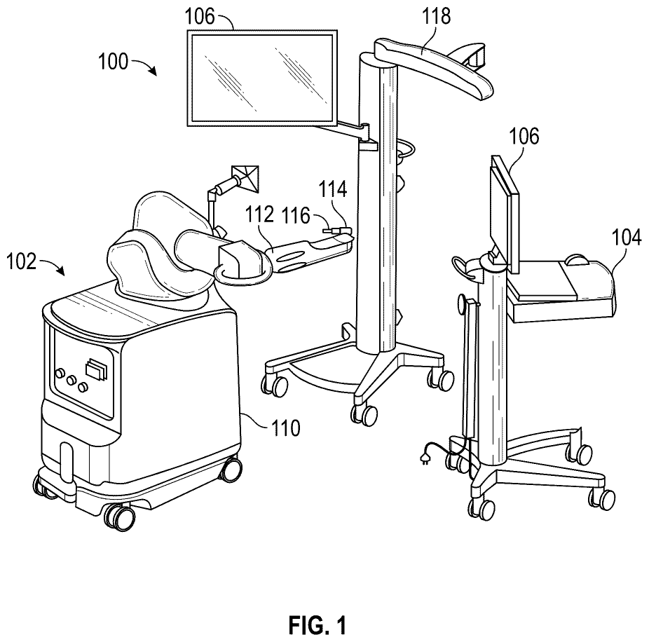

Referring generally to the Figures, exemplary surgical systems and methods are disclosed. As shown in FIG. 1, an exemplary surgical system 100 is shown. The surgical system 100 may be a computer-assisted surgical (CAS) system, a computer-implemented surgical system, and/or any other suitable surgical system. The surgical system 100 may be configured to perform a variety of surgical procedures (e.g., an orthopedic procedure, a total or partial knee arthroplasty procedure, a total or partial hip replacement procedure, a shoulder replacement operation, a knee, hip, or shoulder revision procedure, a hip scoping procedure, a vertebral procedure, a foot or ankle procedure, a hand or wrist procedure, a neurosurgical procedure, a trauma procedure, etc.). It should be understood that the systems described herein (e.g., the surgical system 100, a robotic system, a computing system, etc.), as well as the concepts and processes described herein, may be applicable to any suitable type of surgical procedure (e.g., joint replacement including revision procedures, trauma procedures as for fracture and break repair, sports medicine procedures as for ligament repair, etc.). In some embodiments, the surgical system 100 includes additional, fewer, and/or different components (e.g., a surgical bed, a surgical lighting device, a tracking system, a tracking device, etc.).

As shown in FIG. 1, the surgical system 100 includes a robotic system 102, a computing system 104, and at least one display device, shown as display device 106. The robotic system 102 may include a base 110, an arm 112, a force system, and a controller. In some embodiments, the arm 112 is an articulated arm; however, in other embodiments, the arm 112 is another suitable type of arm (e.g., a telescoping arm, etc.). The robotic system 102 may further include a surgical tool 114. In some embodiments, the surgical tool 114 includes an end effector (e.g., coupled to the arm 112), shown as cutting tool 116. The cutting tool 116 may include an operating member or cutting feature, such as a saw, reamer, burr, or other suitable operating member (e.g., an ultrasonic cutting tool, a water jet, a vibrating tool, etc.). In an exemplary embodiment, the robotic system 102 is used in an interactive manner (e.g., by a healthcare provider, a surgeon, a user, an operator, etc.) to implement or perform a surgical procedure or operation. For example, the robotic system 102 (e.g., the arm 112, the surgical tool 114, the cutting tool 116, etc.) may be manipulated, such that the arm 112 and/or the surgical tool 114 (e.g., the cutting tool 116, etc.) is/are manipulated (e.g., moved, repositioned, etc.) to implement or perform the surgical procedure.

As described herein, the computing system 104 may include hardware and/or software components (e.g., one or more processors, one or more memory devices, computer-readable instructions stored on one or more memory devices, etc.), for example to implement or perform the one or more operations described herein. For example, the computing system 104 may be configured to determine a position and/or orientation associated with one or more objects (e.g., features, devices, components, etc.) within a surgical environment. The computing system 104 may be configured to determine a position and/or orientation associated with one or more objects, for example to implement or perform a surgical procedure and/or plan a surgical procedure. In some embodiments, the computing system 104 may include surgical planning and/or surgical assistance software, which may be used to implement and/or perform a surgical procedure. For example, the computing system 104 may be configured to store one or more images, videos, models (e.g., a three-dimensional model, a virtual model, etc.), which may be used to implement and/or plan a surgical procedure, and/or may be displayed on the display device 106.

In an exemplary embodiment, the computing system 104 is communicably coupled with the robotic system 102 and/or the display device 106. It should be understood that in some embodiments one or more components of the surgical system 100 is/are combined and/or implemented in one or more devices. For example, in some embodiments the robotic system 102, the computing system 104, and/or the display device 106 is/are implemented in a single component or device (e.g., the robotic system 102).

As described herein, the surgical system 100 is configured to determine a pose (e.g., a position, orientation, a position and orientation, etc.) of one or more objects in a surgical environment. For example, the robotic system 102 and/or the computing system 104 may be configured to determine a pose of one or more objects in a space (e.g., a surgical environment, etc.). Further, the surgical system 100 may be configured to establish one or more boundaries (e.g., a haptic region, a haptic restraint, etc.) in a surgical environment. For example, the robotic system 102 and/or the computing system 104 may be configured to determine one or more virtual boundaries using one or more objects in space (e.g., a surgical environment, etc.). In addition, the surgical system 100 may be configured to establish a coordinate frame of reference in a surgical environment. For example, the robotic system 102 and/or the computing system 104 may be configured to establish a coordinate frame of reference using one or more objects in space (e.g., a surgical environment, etc.).

According to an exemplary embodiment, the robotic system 102 (e.g., the arm 112, the surgical tool 114, an end effector, etc.) and/or the computing system 104 is configured to determine a pose of one or more objects in a space, for example a component of the surgical system 100 (e.g., the cutting tool 116). As described herein, an object may include, for example, a tool, an instrument, a patient anatomy, an implant, a prosthetic device, a point in space, a surface (e.g., of a patient anatomy, an implant, a prosthetic device, etc.), and/or one or more components of the surgical system 100. In an exemplary embodiment, the robotic system 102 and/or the computing system 104 is configured to determine a pose of an object via a pose of the cutting tool 116. The robotic system 102 and/or the computing system 104 may include sensors, joint actuators, joint controllers, encoders (e.g., at joints of the arm 112), and/or other mechanical and/or computing components (e.g., an optical tracking system, or a tracking system 118, etc.), configured to determine (e.g., establish, identify, etc.) a pose of the cutting tool 116. For example, the robotic system 102 may also include the tracking system 118, which may be used to detect (e.g., optically, etc.) a pose of the cutting tool 116, either directly or indirectly, for example my detecting a pose of one or more components (e.g., an array, etc.) of the base 110, the arm 112, the surgical tool 114, and/or the cutting tool 116.

Based on one or more poses of the cutting tool 116, the robotic system 102 and/or the computing system 104 may be configured to identify (e.g., associate, determine, establish, etc.) a pose of one or more objects in space. It should be understood that while the surgical system 100 is described herein as identifying (e.g., determining, establishing, etc.) a pose of an object using a pose of the cutting tool 116, it is contemplated that the surgical system 100 may determine a pose of an object using another suitable component, device, and/or system (e.g., a detection device, a pose of the arm 112, a tracking system, etc.).

As discussed above, in an exemplary embodiment the surgical system 100 (e.g., the robotic system 102, the computing system 104, etc.) is configured determine a pose of one or more objects in a space. In some embodiments, the robotic system 102 and/or the computing system 104 is configured to identify an object with respect to a coordinate frame of reference, for example to determine a pose (e.g., a position, orientation, etc.) of the identified object (e.g., relative to the frame of reference, etc.). In some embodiments, the robotic system 102 is configured to identify an object with respect to a coordinate frame of reference, for example to track and/or detect movement of the identified object (e.g., an anatomical feature of a patient, an implant, etc.). In some embodiments, as the cutting tool 116 moves in the coordinate frame of reference, the surgical system 100 (e.g., the robotic system 102, the computing system 104, etc.) can identify one or more objects (e.g., via a pose of the cutting tool 116). The one or more identified objects can be tracked (e.g., monitored, determined, identified, etc.), for example to determine a pose (e.g., a position, orientation, etc.) and/or track movement of the identified object or objects. As a result, the robotic system 102 (and/or the computing system 104) may be configured to capture and/or obtain data associated with a pose and/or movement of an identified object or objects in space (e.g., a surgical environment).

As discussed above, in an exemplary embodiment the surgical system 100 is also configured to register coordinates in a space or environment. For example, the robotic system 102 and/or the computing system 104 may be configured to register (e.g., map, associate, etc.) coordinates in a space or environment, for example to determine a spatial alignment or correspondence between one or more objects in the space or environment (e.g., determine or establish a coordinate frame, for example through a coordinate transformation process, etc.). Objects in physical space may be registered to any suitable coordinate system, for example a coordinate system being used by the robotic system 102 (e.g., a controller, etc.) and/or the computing system 104 (e.g., a controller, processor, memory device, etc.), as discussed herein. In some embodiments, the surgical system 100 (e.g., object data from the robotic system 102, the computing system 104, etc.) uses the object data to establish a coordinate frame of reference (e.g., relative to the object, relative to one or more objects, etc.). For example, the surgical system 100 may establish a coordinate frame of reference using one or more identified objects (e.g., an identified object defining a center of the coordinate frame, a plurality of identified objects defining one or more axis or planes of the coordinate frame, a virtual point spaced a distance from an identified object, etc.).

In some embodiments, the surgical system 100 uses object data to associate a physical anatomy of a patient (e.g., a patient's tibia, etc.) with a coordinate frame of reference. For example, the surgical system 100 may establish a coordinate frame of reference using a first identified object (e.g., an object defining a center of the coordinate frame, etc.), and associate a physical anatomy of a patient using a second identified object (e.g., a patient's tibia using an identification of the lateral condyle, the medial condyle, the tibial tuberosity, etc.). In other embodiments, the surgical system 100 also uses object data to associate a physical anatomy of patient (e.g., a patient's tibia, etc.) with a representation of the anatomy (e.g., an image of the physical anatomy, a virtual model of the physical anatomy, etc.), for example by performing a best fit of points collected by tracking the pose of an object (e.g., cutting tool, probe, etc.) by the surgical system 100 with a surface of a model of the patient's anatomy (e.g., based on pre-operative CT or other imaging). In some embodiments, the surgical system 100 is configured to establish a coordinate frame of reference, associate a physical anatomy of a patient relative to a coordinate frame, associate a representation of the anatomy relative to a coordinate frame, and/or any combination thereof (e.g., using object data). In this regard, using the object data (e.g., an identified object, a tracked object, etc.), the surgical system 100 may determine one or more spatial relationships between a coordinate frame, an anatomy of a patient, a representation of an anatomy of a patient, and/or a combination thereof.

In an exemplary embodiment, registration includes any suitable registration technique. For example, the registration technique may include physical-space registration (e.g., where a patient's actual anatomy is registered relative to a coordinate frame), image-to-image registration (e.g., monomodal registration where images of the same type or modality, such as fluoroscopic images or MR images, are registered and/or multimodal registration where images of different types or modalities, such as MRI and CT, are registered), image-to-physical space registration (e.g., image-to-patient registration where a digital data set of a patient's anatomy obtained by conventional imaging techniques is registered with the patient's actual anatomy), combined image-to-image and image-to-physical-space registration (e.g., registration of preoperative CT and MRI images to an intraoperative scene), and/or registration using a video camera, ultrasound, and/or another suitable system or device (e.g., a tracking system, etc.). The surgical system 100 (e.g., the robotic system 102, the computing system 104, etc.) may also include a coordinate transform process for mapping (or transforming) coordinates in one space to those in another to achieve spatial alignment or correspondence. For example, the surgical system 100 may use the coordinate transform process to map positions of identified objects (e.g., an identified object for registration, an identified object for coordinate frame registration, an identified object on a patient anatomy, an identified object to determine a virtual boundary, etc.) into a coordinate system used by a process running on the computer of a haptic device and/or a surgical controller. The coordinate transform process may include any suitable transformation technique, such as, for example, rigid-body transformation, non-rigid transformation, affine transformation, and the like. In some embodiments, a camera and/or a scan of patient anatomy can be used to obtain a model and/or register the model. For example, an initial 3D model can be created and registered. In some embodiments, a video camera can be used to register a 3D model corresponding to a CT scan. According to some embodiments, a video camera, ultrasound, and/or another suitable system or device (e.g., a tracking system, etc.) can be used for both initial model creation and/or registration.

As discussed above, in an exemplary embodiment the surgical system 100 is also configured to determine one or more boundaries in space (e.g., a surgical environment, etc.). For example, the robotic system 102 and/or the computing system 104 may be configured identify an object (e.g., an object in space, an anatomy of a patient, etc.) with respect to a coordinate frame of reference, for example to determine a pose (e.g., a position, etc.) of the identified object (e.g., relative to the frame of reference). In some embodiments, the robotic system 102 (e.g., via a pose of the cutting tool 116, etc.) is configured to identify an object (or objects) with respect to a coordinate frame of reference, for example to establish one or more virtual boundaries in space. As described herein, the one or more virtual boundaries may provide a guide or restraint (e.g., a boundary, a limit, etc.), for example to control or guide manipulation of a surgical tool (e.g., the cutting tool 116, etc.). In this regard, the surgical system 100 (e.g., the robotic system 102, the computing system 104, the force system, etc.) may be implemented with one or more virtual boundaries, for example to control or guide manipulation of a surgical tool during a surgical operation or procedure.

In an exemplary embodiment, the surgical system 100 (e.g., the force system, controller, etc.) is configured to provide a cutting restraint guide via control or guidance to the surgeon during manipulation of the surgical tool 114 (e.g., the cutting tool 116, etc.). The force system (e.g., motors operating joints of the arm 112) may be configured to provide at least some force to the surgical tool 114 via the arm 112, and a controller may be programmed to generate control signals for controlling the force system. In some embodiments, the force system includes actuators and a back-driveable transmission that provide haptic (or force) feedback to constrain or inhibit the surgeon from moving the surgical tool beyond predefined haptic boundaries defined by haptic objects as described, for example, in U.S. Pat. No. 8,010,180 and/or U.S. patent application Ser. No. 12/654,519 (U.S. Patent Application Pub. No. 2010/0170362), filed Dec. 22, 2009, each of which is hereby incorporated by reference herein in its entirety. As described herein, the force system and/or the controller may be housed within the robotic system 102 and/or the computing system 104. In some embodiments, cutting restraint or guidance is provided though a handheld manipulator or handheld robotic device, such as described in U.S. Pat. No. 9,399,298 entitled “Apparatus and Method for Providing an Adjustable Positive Stop in Space,” U.S. Pat. No. 9,060,794 entitled “System and Method for Robotic Surgery,” and U.S. Patent Publication No. 2013/0060278 entitled “Surgical instrument including housing, a cutting accessory that extends from the housing and actuators that establish the position of the cutting accessory relative to the housing,” each of which is incorporated herein by reference in its entirety.

According to an exemplary embodiment, the surgical system 100 is configured to continually determine (e.g., track, etc.) a pose of any relevant and/or identified object (e.g., an anatomy of a patient, a prosthetic, a boundary, etc.). For example, the surgical system 100 (e.g., the robotic system 102, etc.) may include non-mechanical tracking components, mechanical tracking components, and/or any combination of non-mechanical and mechanical tracking components suitable for use in a surgical environment. The non-mechanical tracking components may include virtual, optical (or visual), magnetic, radio, or acoustic tracking components. Such components are configured to be associated with (e.g., appointed to, assigned to, etc.) an object to be tracked (e.g., a point in space, an anatomical feature, an anatomical surface, etc.) and/or may be an inherent component of the object to be tracked (e.g., an identifiable anatomical feature, an identifiable prosthetic feature, etc.). For example, a trackable element (e.g., a virtual trackable element, etc.) may include an array of objects identified on a surface of a patient's anatomy (e.g., via a pose of the cutting tool 116), which may define a geometric arrangement and/or a geometric relationship of the trackable element relative a patient's anatomy. In some embodiments, trackable elements (e.g., a virtual trackable element) include objects identified on a patient's anatomy (e.g., via a pose of the cutting tool 116), which may define a known geometric arrangement of a particular patient's anatomy (e.g., relative to a defined coordinate frame, etc.). Thus, the surgical system 100 can recognize a particular identified object (e.g., a marked or tracked object, etc.), at least in part, from the geometry of the markers, an orientation of the axis, and/or a location of the endpoint within a frame of reference deduced from positions of the markers.

As described herein, the trackable marker (e.g., identified objects, etc.) may include any known marker, for example intrinsic features of an identified object (e.g., a tracked object). For example, the markers may include intrinsic features that are salient and/or accurately locatable portions of objects sufficiently defined and/or identifiable to function as recognizable markers (e.g., anatomical landmarks, outlines of anatomical structures, shapes, outlines of and/or on anatomical features, etc.). In some embodiments, the markers are extrinsic markers (e.g., markers affixed to skin, markers implanted in bone, fiducial arrays, stereotactic frames, etc.) designed to be accurately detectable by the surgical system 100. According to an exemplary embodiment, the markers (e.g., identified objects) are identified (e.g., determined, tracked, located, etc.) using any suitable detection method. For example, the markers may be identified (e.g., determined, established, etc.) via a pose of one or more identified objects (e.g., an object identified by the robotic system 102 via a pose of the cutting tool 116, etc.). In some embodiments, the markers are identified using a tracking device or system, for example the tracking system 118, which may be used to detect a pose of the markers (e.g., directly, indirectly via a pose of one or more components, etc.). In some embodiments, the markers are identified using another suitable system or device (e.g., a detection device, a tracking system, an optical tracking system, a magnetic tracking system, etc.).

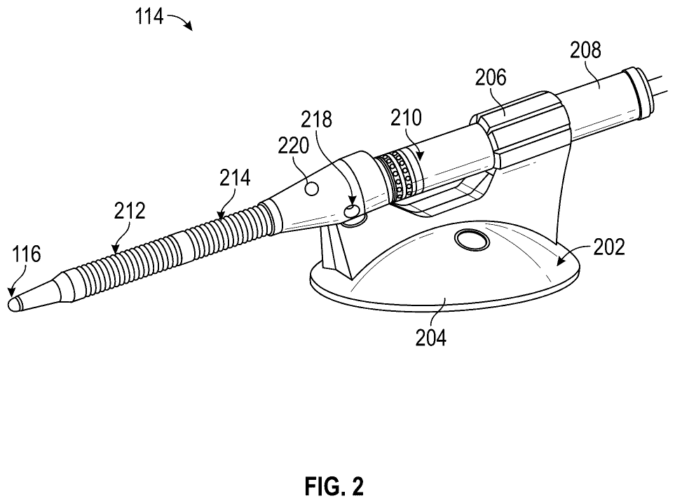

Referring now to FIG. 2, the surgical tool 114 is shown in greater detail, according to an exemplary embodiment. Various different surgical tools can be used in various embodiments of the teachings herein (e.g., burrs, reamers, saws, drills, ultrasonic tools, laser ablation tools, etc.). As shown in FIG. 2, the surgical tool includes a housing 202 with a base 204 and a mount 206. The base 204 may be configured to secure the surgical tool 114 to the arm 112, for example to provide stability to the surgical tool 114. Further, the mount 206 may be configured to secure a shaft 208 of the surgical tool 114. The shaft 208 may include a motor 210 that provides power to the cutting tool 116 located at a distal end of the surgical tool 114. In some embodiments, the surgical tool 114 includes a suction hole 218. The suction hole 218 may connect to an internal channel within the shaft 208. The internal channel may be configured to receive components of the cutting tool 116 (e.g., a cutting burr, etc.). In other embodiments, the internal channel is used as an irrigation or suction channel.

According to an exemplary embodiment, the surgical tool 114 further includes a button 220. The button 220 may be manipulatable (e.g., pressed, pushed, etc.), for example to cause the surgical system 100 to capture (e.g., determine, identify, etc.) a pose (e.g., position, configuration, etc.) of a component of the surgical tool 114 (e.g., the cutting tool 116). In some embodiments, the button 220 includes haptic response capabilities (e.g., causes capture of a pose of the cutting tool 116 based on one or more taps, pattern of taps, etc.), audible capabilities (e.g., causes capture of a pose of the cutting tool 116 based on a voice command, other sound, etc.), motion capture capabilities (e.g., causes capture of a pose of the cutting tool 116 based on a movement of a user's anatomy, e.g., finger, across the button 220), and/or other suitable capabilities. In other embodiments, the button 220 may be used to receive an instruction from a user to capture (e.g., determine, identify, etc.) a pose (e.g., position, configuration, etc.) of another component of the surgical system 100 (e.g., the arm 112, the surgical tool 114, a detection device, etc.).

In an exemplary embodiment, the surgical tool 114 further includes at least one bending element. For example, the surgical tool 114 is shown to include a first bending element 212 and a second bending element 214. The bending elements 212, 214 each include two degrees of freedom that can be bent less or over 90 degrees in a three-dimensional space. Further, as described herein the cutting tool 116 may include one or more tools or devices (e.g., for cutting a type of tissue, bone, etc.). For example, the cutting tool 116 may include a saw (e.g., for a planar cut), a burr (e.g., for a curved surface), a curved saw (e.g., to obtain access around pegs, keels and/or screws, and/or another suitable cutting tool. In some embodiments, the cutting tool 116 is an ultrasonic tool (e.g., used to vibrate and/or break up bone cement), a laser tool (e.g., to melt cement, etc.) a waterjet (e.g., to break up cement, etc.), and/or another suitable cutting tool. The first bending element 212 and the second bending element 214 can include one or more sensors (e.g., fiber optic sensors, strain gauge, etc.) configured to measure a bending of the elements 212, 214 so as to provide data indicative of a relative position of a tip of the cutting tool 116 relative to a body of the surgical tool 114 (e.g., relative to the base 204, relative to the arm 112 on which the base 204 is mounted, etc.). In other embodiments, the surgical tool 114 is provided as a rigid body such that a geometric relationship between the cutting tool 116 and the base 204 (and the arm 112 when base 204 is mounted on the arm 112) is static and known to the surgical system 100.

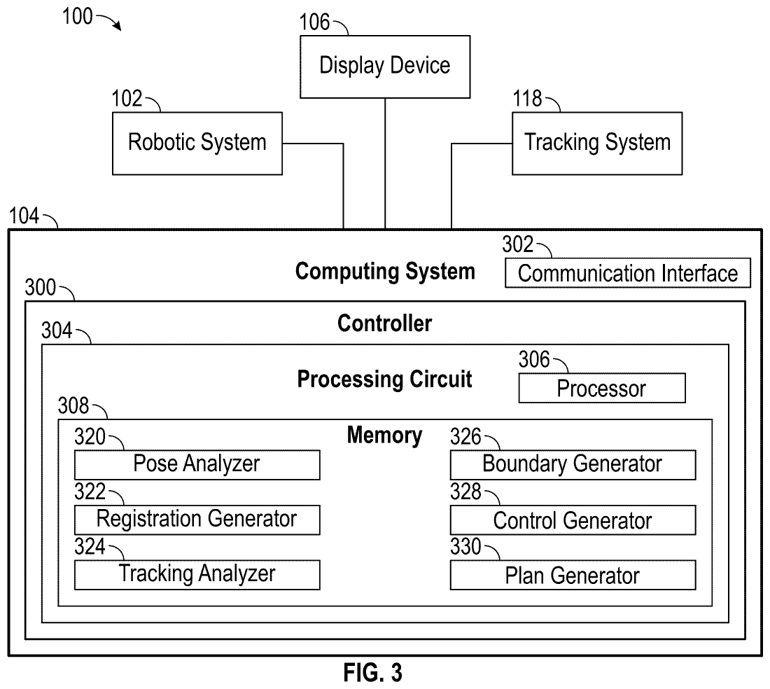

Referring now to FIG. 3, a block diagram of the surgical system 100 is shown, according to an exemplary embodiment. As discussed above, the surgical system 100 may be configured to identify a pose of one or more identified objects in space. For example, the surgical system 100 may be configured to identify a pose of one or more objects in space, for example to establish a coordinate frame of reference in a surgical environment, determine one or more virtual boundaries in the surgical environment, track a position and/or movement of one or more objects in the surgical space, and/or a combination thereof.

As shown in FIG. 2, the computing system 104 may be communicably connected to the robotic system 102, the display device 106, and/or the tracking system 118. It should be understood that some, or all, of the components of the computing system 104, the robotic system 102, and/or the display device 106 may be integrated within a single device, or distributed across multiple separate systems or devices. In some embodiments, components of the computing system 104, the robotic system 102, and/or the display device 106 are components of a controller, are implemented as part of a cloud-based computing system, and/or are part of another suitable system or device that receives, processes, and/or communications data from/to devices or other sources.

As shown, the computing system 104 includes a controller 300 and a communications interface 302. The controller 300 is shown to include and a processing circuit 304 having a processor 306 and a memory 308. The communications interface 302 may include wired or wireless communications interfaces (e.g., ports, jacks, antennas, transmitters, receivers, transceivers, wire terminals, etc.) for communicating data between the computing system 104 and external systems or devices (e.g., the robotic system 102, the display device 106, etc.). In some embodiments, the communications interface 302 facilitates communications between the computing system 104 and one or more external applications and/or interfaces (e.g., a user application, a provider application, etc.), for example to allow a remote user or operator to control, monitor, and/or adjust components of the computing system 104. Further, the communications interface 302 may be configured to communicate with external systems and/or devices using any of a variety of communications protocols (e.g., HTTP(S), WebSocket, CoAP, MQTT, etc.) and/or any of a variety of other protocols. Advantageously, the computing system 104 may obtain, ingest, and process data from any type of system or device, regardless of the communications protocol used by the system or device.

The processor 306 may be a general purpose or specific purpose processor, an application specific integrated circuit (ASIC), one or more field programmable gate arrays (FPGAs), a group of processing components, or other suitable processing components. The processor 306 may further be configured to execute computer code or instructions stored in the memory 308 or received from other computer readable media (e.g., USB or other local storage, network storage, a remote server, etc.).

The memory 308 may include one or more devices (e.g., memory units, memory devices, storage devices, etc.) for storing data and/or computer code for completing and/or facilitating the various processes described in the present disclosure. The memory 308 may include random access memory (RAM), read-only memory (ROM), hard drive storage, temporary storage, non-volatile memory, flash memory, optical memory, or any other suitable memory for storing software objects and/or computer instructions. In some embodiments, the memory 308 may include database components, object code components, script components, and/or any other type of information structure for supporting the various activities and information structures described in the present disclosure. The memory 308 may be communicably connected to the processor 306 via the processing circuit 304, and may include computer code for executing (e.g., by the processor 306) one or more processes described herein. When the processor 306 executes instructions stored in the memory 308, the processor 306 may configure the processing circuit 304 to complete such activities.

As shown, the computing system 104 (e.g., the memory 308) includes a pose analyzer 320, a registration generator 322, a tracking analyzer 324, a boundary generator 326, a control generator 328, and a plan generator 330. The following paragraphs describe some of the functions performed by each of the components 320-330 of the computing system 104, for example operations provided via execution of one or more instructions stored by the components 320-330 of the memory 308 by the processor 306. It should be noted that the number and types of components are merely illustrative, and in some embodiments, implementations of the computing system 104 may have additional, fewer, and/or different components than those illustrated in FIG. 3.

According to an exemplary embodiment, the pose analyzer 320 is configured to obtain input data, analyze the input data, and/or generate output data to be communicated to other components of the computing system 104. For example, the pose analyzer 320 may obtain (e.g., receive, request, pull, etc.) pose data. As described herein, the pose data may be received from and/or associated with a system or device (e.g., the cutting tool 116 of the surgical tool 114, the robotic system 102, a database or storage device, etc.), for example via the communications interface 302.

In an exemplary embodiment, the pose data identifies a pose (e.g., a position, an orientation, a position and orientation, etc.) of one or more objects in space. As discussed herein, the object may include, for example, a tool, an instrument, a patient anatomy, an implant, a prosthetic device, a point in space, a virtual boundary, a surface, and/or another suitable component of the surgical system 100. According to an exemplary embodiment, a pose of the object is captured via a pose of the cutting tool 116. For example, the cutting tool 116 may be used to establish (e.g., determine, identify, etc.) and/or create a marker or tracker (e.g., a virtual marker, a virtual tracker, etc.) associated with the object (e.g., an anatomy of a patient, a point in space, a virtual boundary, etc.), where the marker or tracker is used to subsequently identify a pose of the object. In some embodiments, the pose analyzer 320 (e.g., via a pose of the cutting tool 116, via data from the robotic system 102, etc.) establishes and/or creates a marker or tracker in response to an input. For example, the pose analyzer 320 may establish a marker on an anatomy of a patient (e.g., a virtual marker on a patient's lateral condyle) in response to a user or operator manipulating the button 220.

As will be discussed in greater detail below, pose data may include position and/or information relating to one or more objects in a space. For example, the pose data may identify a pose (e.g., a position, etc.) of an anatomical landmark of an anatomy of a patient (e.g., a lateral condyle of a tibia of a patient). The pose data may trace an anatomical surface of a patient, for example to register and/or associate an anatomy of a patient (e.g., a joint, a tibia, etc.) relative to a coordinate frame. In some embodiments, the pose data traces an anatomical surface of a patient, for example to generate a virtual boundary associated with the anatomical surface. In other embodiments, the pose data traces a virtual boundary, for example to establish a virtual cutting boundary. In other embodiments, the pose data identifies an object (e.g., a point in space, a point on or in a patient's anatomy, etc.), for example to establish a center of a coordinate frame. In this regard, the pose analyzer 320 may be configured to receive and/or process pose data relating to one or more objects in space (e.g., via pose data associated with the cutting tool 116, etc.), which may be communicated to other components of the computing system 104 for example to establish a coordinate frame of reference in a surgical environment, determine one or more virtual boundaries in the surgical environment, track a position and/or movement of one or more objects in the surgical space, and/or a combination thereof.

According to an exemplary embodiment, the registration generator 322 is configured to obtain input data, analyze the input data, and/or generate output data to be communicated to other components of the computing system 104. For example, the registration generator 322 may be configured to obtain (e.g., receive, request, pull, etc.) registration data. The registration data may be, or include, pose data (e.g., from the pose analyzer 320, from another system or device, for example the robotic system 102 (e.g., via a pose of the cutting tool 116, etc.), as discussed herein. In some embodiments, the registration generator 322 is configured to obtain registration data, analyze the registration data, and/or register one or more objects in a space.

In an exemplary embodiment, the registration generator 322 is configured to analyze registration data (e.g., including a pose, pose data, etc.), and register one or more objects. For example, the registration generator 322 may obtain registration data that includes a pose (e.g., position, etc.) of an identified point in space. The registration generator 322 may be configured to analyze the pose, and generate or establish a coordinate frame relative to the identified point in space (e.g., with the identified point as a center of the coordinate frame, etc.). In other embodiments, the registration generator 322 is configured to receive registration data that includes a plurality of poses (e.g., positions, etc.) of identified points in space (e.g., three identified points in three-dimensional space, etc.). The registration generator 322 may be configured to analyze the poses, and generate or establish a coordinate frame relative to the identified points in space (e.g., a coordinate frame with a first plane fit to two of the identified points, a second plane fit to one of the identified points that intersects with the first plane, etc.). In some embodiments, the registration generator 322 is configured to obtain registration data that includes a pose (e.g., a position) of an identified anatomical feature of a patient (e.g., a lateral condyle of a tibia of a patient, etc.). The registration generator 322 may be configured to analyze the data, and generate or establish a coordinate frame relative to the identified point (e.g., with the identified point as a center of the coordinate frame, with a virtual point spaced apart from the identified point as a center of the coordinate frame, etc.). In other embodiments, the registration generator 322 is configured to obtain registration data, analyze the registration data, and/or generate or otherwise establish a coordinate frame based on the registration data (e.g., about an identified object, about a virtual point spaced apart from the identified object, etc.).

In an exemplary embodiment, the registration generator 322 is also configured to obtain registration data, analyze the registration data, and/or register one or more tools, instruments, devices, anatomical features, prosthetics, and/or other suitable objects (e.g., markers, trackers, etc.). For example, the registration generator 322 may obtain registration data that includes a pose (e.g., position, etc.) of an identified anatomical feature of a patient (e.g., a lateral condyle of a tibia of a patient, etc.). The registration generator 322 may be configured to analyze the data, and associate and/or establish (e.g., register) the anatomy of the patient relative to a coordinate frame (e.g., the coordinate frame established via the registration data). In some embodiments, the registration generator 322 is configured to obtain registration data that includes a plurality of poses of one or more features (e.g., a lateral condyle of a tibia of a patient, a connection surface of an implant, etc.), analyze the data, and associate and/or establish (e.g., register) the features relative to a coordinate frame. In some embodiments, the registration generator 322 is configured to obtain registration data in sequence. For example, during a surgical procedure or operation, the registration generator 322 may obtain registration data that includes sequential poses (e.g., positions, a checkpoint, etc.) of an identified anatomical feature of a patient (e.g., a lateral condyle of a tibia of a patient, etc.). The registration generator 322 may be configured to analyze the data, and associate and/or establish (e.g., register) the anatomy of the patient relative to a coordinate frame, for example to determine whether a change in pose has occurred (e.g., a tibia of a patient has moved or repositioned, an implant has moved or shifted, etc.) relative to subsequent poses (e.g., via the checkpoint, etc.). In this regard, the registration generator 322 may be configured to receive and/or analyze registration data (e.g., pose data associated with a checkpoint, a pose of one or more identified objects, etc.), for example to establish a coordinate frame of reference in a surgical environment, to register and/or associate relative poses of features in the surgical environment (e.g., an anatomy of a patient, an implant, etc.), and/or monitor poses of the features in the surgical environment over the course of a surgical procedure.

According to an exemplary embodiment, the tracking analyzer 324 is configured to obtain input data, analyze the input data, and/or generate output data to be communicated to other components of the computing system 104. For example, the tracking analyzer 324 may be configured to obtain (e.g., receive, request, pull, etc.) tracking data. The tracking data may be, or include, pose data (e.g., from the pose analyzer 320, etc.), or from another system or device, for example the robotic system 102 (e.g., via a pose of the cutting tool 116, etc.), as discussed herein. In some embodiments, the tracking analyzer 324 is configured to obtain tracking data, analyze the tracking data, and/or track or monitor one or more objects in a space.

In an exemplary embodiment, the tracking analyzer 324 is also configured to obtain tracking data, analyze the tracking data, and/or track one or more tools, instruments, devices, anatomical features, prosthetics, and/or other suitable objects (e.g., markers, trackers, etc.). For example, the tracking analyzer 324 may obtain tracking data that identifies (e.g., marks, etc.) a pose (e.g., a position, etc.) of an anatomical landmark of an anatomy of a patient (e.g., a lateral condyle of a tibia of a patient), for example relative to a coordinate frame (e.g., the coordinate frame established via the registration generator 322, etc.). The tracking analyzer 324 may be further configured to monitor and/or track a pose of the anatomy of the patient (e.g., via a virtual marker or tracker, via a checkpoint, etc.), for example to track or monitor movement of the patient. In some embodiments, the tracking analyzer 324 is configured to obtain tracking data that identifies (e.g., marks, etc.) a plurality of features (e.g., an anatomy of a patient, a contact point of an implant, an anatomical surface of a patient, etc.), for example relative to a coordinate frame. The tracking analyzer 324 may further be configured to monitor and/or track a pose of the features, for example to track or monitor movement of the features. In other embodiments, the tracking analyzer 324 is configured to obtain tracking data that identifies (e.g., marks, etc.) a pose of an identified point (e.g., a point in space relative to an anatomy of a patient, a point in space relative to an implant, a virtual point spaced apart from an identified point in space, a virtual point spaced apart from an identified point in space in an anatomy of a patient, etc.). The tracking analyzer 324 may further be configured to monitor and/or track a pose of the identified point, for example to track or monitor movement of the point (e.g., within a coordinate frame, relative to other anatomy of a patient etc.). In this regard, the tracking analyzer 324 may be configured to obtain and/or analyze tracking data, for example track or monitor movement of an anatomy of a patient, a feature (e.g., an implant, a surface, a tool, an instrument, etc.) in a surgical environment (e.g., coordinate frame, etc.), and/or an identified point or object.

According to an exemplary embodiment, the boundary generator 326 is configured to obtain input data, analyze the input data, and/or generate output data to be communicated to other components of the computing system 104. For example, the boundary generator 326 may be configured to obtain (e.g., receive, request, pull, etc.) boundary data. The boundary data may be, or include, pose data (e.g., from the pose analyzer 320, etc.), from another system or device, for example the robotic system 102 (e.g., via a pose of the cutting tool 116, etc.), as discussed herein. In some embodiments, the boundary generator 326 is configured to obtain boundary data, analyze the boundary data, and/or generate one or more virtual boundaries.

In an exemplary embodiment, the boundary generator 326 is configured to analyze boundary data (e.g., including a pose, pose data, etc.), and generate one or more virtual boundaries. As discussed herein, a virtual boundary may establish or provide a guide or restraint (e.g., a boundary, a limit, a threshold, etc.), for example to control or guide manipulation of a tool or instrument in a surgical field.

For example, the boundary generator 326 may obtain boundary data that includes pose data (e.g., position, configuration, etc.) that traces a surface of an anatomy of a patient (e.g., a plurality of positions that trace a surface, via a plurality of positions of the cutting tool 116, etc.). The boundary generator 326 may be configured to analyze the data, and generate a virtual boundary. The virtual boundary may be or include a projection based on the pose data. For example, the boundary generator 326 may be configured to establish a plane that includes or is best-fit to a plurality of positions (e.g., 3, 4, 5, 50, 100, etc. positions) that trace the surface (or a portion thereof). Based on the plane, the boundary generator 326 may be configured to determine an orientation relative to the plane (e.g., 5, 10, 15, 30, 45, 60, 75, 80, 90, 100, etc. degrees relative to the plane), and project a virtual boundary a distance from the plane, for example in the direction of the determined orientation. In an exemplary embodiment, the distance is established via an input (e.g., a user or operator input, etc.), via a setting (e.g., a user or operator setting, a manufacturer setting, etc.), via a surgical planning system (e.g., via a surgical plan, a treatment plan, a selected implant or therapeutic device, etc.), and/or via another suitable input, setting, and/or system or device. In some embodiments, the distance (e.g., 5 millimeters, 1 centimeter, 2 centimeters, etc.) is associated with a selected cutting depth.

In some embodiments, the boundary generator 326 obtains boundary data that includes pose data (e.g., position, configuration, etc.) that traces a line relative to an anatomy of a patient (e.g., a plurality of positions that trace a line, etc.). The boundary generator 326 may be configured to analyze the data, and generate a virtual boundary. The virtual boundary may be or include a projection based on the pose data. For example, the boundary generator 326 may be configured to establish (e.g., fit, etc.) a line that includes a plurality of the positions (e.g., 3, 4, 5, 50, 100, etc. positions) that trace the line (or a portion thereof). Based on the line, the boundary generator 326 may be configured to associate the line within a coordinate frame (e.g., the coordinate frame established by the registration generator 322, etc.), and project a virtual boundary a distance (e.g., user-selected distance) from the line (e.g., in a vertical direction relative to the coordinate frame, at an angle relative to an axis or plane of the coordinate frame, etc.). In some embodiments, the distance is associated with a user-selected cutting depth, and/or establishes a cutting boundary relative to an anatomy of a patient, as discussed herein. In other embodiments, the boundary generator 326 is configured to project a virtual boundary a distance from the line using additional pose data (e.g., an identified anatomical structure of the patient, a component of an implant, etc.). For example, the boundary generator 326 may be configured to project a virtual boundary a distance from the line in a direction relative to the identified anatomical structure (e.g., toward the identified anatomical structure, angled relative to the identified anatomical structure, etc.) within a coordinate frame.

In some embodiments, the boundary generator 326 obtains boundary data that includes pose data (e.g., position, configuration, etc.) that identifies a plurality of objects (e.g., points, anatomical features, points on an implant, etc.). The boundary generator 326 may be configured to analyze the data, and generate a virtual boundary. The virtual boundary may be or include a projection based on the pose data, and/or the virtual boundary may be or include one or more predefined shapes or configurations. For example, the boundary generator 326 may analyze the plurality of objects (e.g., points, anatomical features, etc.), and generate a virtual boundary that is a cone or pyramid defined by the plurality of objects (or a portion thereof). For example, the virtual boundary (e.g., cone or pyramid shape) may be defined such that the plurality of objects define a base of the virtual boundary (e.g., the cone, pyramid, etc.), and a projection (e.g., to a selected cutting depth, a desired distance, etc.) defines an apex of the virtual boundary. In some embodiments, the virtual boundary (e.g., cone or pyramid shape, etc.) is defined such that a portion of the plurality of objects define a base of the virtual boundary (e.g., the cone, pyramid, etc.), and one of the plurality of objects (e.g., an identified anatomical feature, an identified or projected point in space, etc.) defines an apex of the virtual boundary. In other embodiments, the boundary generator 326 is configured to analyze the plurality of objects (e.g., points, anatomical features, points on an implant, etc.), and generate a virtual boundary that is another suitable shape (e.g., sphere, hemisphere, cylinder, cube, triangular prism, square, triangle, circle, etc.). In this regard, it is contemplated herein that any combination of the plurality of objects and/or a distance from one or more of the objects (e.g., a selected cutting depth, a selected distance, etc.) may be used to define one or more surfaces, points (e.g., center point, point of rotation, etc.), planes, axis, and/or edges of the shapes and/or configurations of the virtual boundary (e.g., a base, a side edge, an apex, a portion of a circumference, etc.).

In some embodiments, the boundary generator 326 obtains boundary data that includes pose data (e.g., position, configuration, etc.) that identifies a plurality of objects (e.g., points, anatomical features, points on a surface of an anatomical feature, etc.). The boundary generator 326 may be configured to analyze the data, and generate a virtual boundary. The virtual boundary may be or include a projection based on the pose data, and/or the virtual boundary may be or include a shape of an implant. For example, the boundary generator 326 may analyze the plurality of objects (e.g., points, anatomical features, etc.), and generate a virtual boundary that is the shape of a selected implant (e.g., based on the plurality of objects). In some embodiments, the virtual boundary is determined based on one or more virtual implant models stored in a database or other storage device (e.g., a storage device of the computing system 104, a storage device of the robotic system 102, an external or remote storage device). For example, the boundary generator 326 may generate a virtual boundary (e.g., the implant) that uses the plurality of objects as connection points for the implant that is to be implanted. In other embodiments, the boundary generator 326 is configured to generate a virtual boundary that is or includes shapes of other features or components (e.g., bone cement, bone screws, etc.).

In some embodiments, the boundary generator 326 is configured to obtain boundary data (e.g., including one or more points of pose data), analyze the boundary data, and generate a plurality of virtual boundaries. For example, the boundary generator 326 may receive boundary data that includes pose data that identifies a plurality of objects (e.g., points, anatomical features, etc.). Using the boundary data, the boundary generator 326 may be configured to generate a first virtual boundary that includes a predefined shape or configuration (e.g., a cylinder-shaped virtual boundary of selected depth, a cone-shaped virtual boundary of user-selected depth, etc.), and a second virtual boundary that includes a projection of a line based on the boundary data (e.g., a virtual boundary extending vertically from a line in a coordinate plane, etc.). In some embodiments, the plurality of virtual boundaries are layered. For example, the second virtual boundary (e.g., projection of the line) may intersect with the first virtual boundary (e.g., cylinder-shaped virtual boundary of selected depth, etc.), for example to partition or separate the plurality of boundaries. In this regard, in some embodiments the boundary generator 326 may be configured to layer one or more virtual boundaries.

In some embodiments, the boundary generator 326 is configured to obtain boundary data (e.g., including one or more points of pose data), analyze the boundary data, and generate a one or more virtual boundaries that identify a cutting surface or material. For example, the boundary generator 326 may receive boundary data that includes pose data that identifies a plurality of objects (e.g., points, anatomical features, points on an implant, etc.), analyze the boundary data, and generate a virtual boundary, as described herein. In some embodiments, the virtual boundary includes a projection including one or more portions that identify one or more surfaces or materials. For example, the virtual boundary may include a projection that includes a first portion (e.g., a top surface, a top portion of predefined depth, etc.) identifying a first cutting surface as an implant, and a second portion (e.g., a lower or middle portion, etc.) identifying a second cutting surface as bone. In some embodiments, the virtual boundary includes a projection that identifies a first portion (e.g., a middle portion, etc.) identifying a first cutting surface as bone, and a second portion (e.g., a lateral portion, etc.) identifying a second cutting material as anatomical tissue (e.g., cartilage, muscle, etc.). In this regard, in some embodiments the virtual boundary may include a projection that identifies a cutting surface or material of one or more portions of the projection, for example to control or guide use of instruments or tools (e.g., the cutting tool 116) in different areas of the surgical space (e.g., the virtual boundary).

It should be understood that while the components of the computing system 104 (e.g., the pose analyzer 320, the registration generator 322, the tracking analyzer 324, the boundary generator 326, etc.) are described herein as being capable of, among other features, establishing a coordinate frame of reference and/or registering one or more features in a surgical environment, determining one or more virtual boundaries in the surgical environment, tracking a position and/or movement of one or more objects in the surgical space, and/or a combination thereof, it is contemplated that in other embodiments the components of the computing system 104 are configured to perform one or more of the operations described herein independently, in another combination, and/or in any suitable sequence or order. For example, in some embodiments the computing system 104 (e.g., the boundary generator 326) may be configured to generate one or more virtual boundaries (e.g., using pose data) without establishing a coordinate frame and/or registering an anatomical feature of a patient. Further, in some embodiments, the computing system 104 (e.g., the boundary generator 326) may be configured to generate one or more virtual boundaries (e.g., using pose data) without tracking an anatomy of a patient and/or another feature of the surgical system 100. In other embodiments, the computing system 104 (e.g., the registration generator 322) may be configured to establish a coordinate frame of reference and register an anatomy of a patient in a first step of a procedural process (e.g., registration and calibration phase, etc.). The computing system 104 (e.g., the boundary generator 326) may then be configured to generate one or more virtual boundaries (e.g., based on a traced surface of a patient's anatomy), as part of the procedural process (e.g., intraoperative planning workflow, etc.). The computing system 104 (e.g., the tracking analyzer 324) may then be configured to track an anatomy of the patient (e.g., a tibia of the patient), an instrument (e.g., the cutting tool 116), and/or one or more virtual boundaries (e.g., the generated virtual boundary), as part of the procedural process (e.g., a bone cutting phase, anatomical preparation phase, etc.). As such, it should be understood that the computing system 104 may be configured to implement some, any, and/or all of the features described herein, independently, in one or more combinations, and/or in any suitable order or sequence.

According to an exemplary embodiment, the control generator 328 is configured to obtain input data, analyze the input data, and/or generate output data to be communicated to other components of the surgical system 100. For example, the control generator 328 may be configured to obtain (e.g., receive, request, pull, etc.) control data. The control data may be, or include, pose data (e.g., from the pose analyzer 320, etc.), registration data (e.g., from the registration generator 322, etc.), tracking data (e.g., from the tracking analyzer 324, etc.), and/or boundary data or virtual boundary data (e.g., from the boundary generator 326, etc.). In some embodiments, the control data is received from one or more components of the computing system 104, and/or another suitable system or device, for example the robotic system 102 (e.g., via data from the cutting tool 116) and/or a storage device. In an exemplary embodiment, the control generator 328 is configured to obtain control data, analyze the control data, and/or generate one or more control decisions.

In an exemplary embodiment, the control generator 328 is configured to obtain control data, analyze the control data, and generate one or more control decisions. In some embodiments, the control decisions are communicated to one or more components of the surgical system 100 (e.g., the robotic system 102, the display device 106, etc.). The control decisions may include one or more instructions to implement one or more actions (e.g., restraints, restrictions, guidance, control, automated control, etc.). For example, the control decisions may be communicated to the robotic system 102, and may include instructions to guide or control the robotic system 102 (e.g., the arm 112, the surgical tool 114, the cutting tool 116, etc.) during a surgical procedure or operation. In some embodiments, the control decisions include instructions to be implemented by the force system, as described herein. In other embodiments, the control decisions include one or more instructions to implement a communication. For example, the control decisions may be communicated to the display device 106, and may cause the display device 106 to display one or more interfaces (e.g., containing a virtual model, an image, video, or representation of an operation, etc.), instructions or messages, and/or alerts or indicators. In other embodiments, the control decisions include other suitable instructions (e.g., commands, etc.) to implement one or more of the processes and/or functions described herein.

According to an exemplary embodiment, the plan generator 330 is configured to obtain input data, analyze the input data, and/or generate output data to be communicated to other components of the surgical system 100. For example, the plan generator 330 may be configured to obtain (e.g., receive, request, pull, etc.) planning data. The planning data may be, or include, pose data (e.g., from the pose analyzer 320, etc.), registration data (e.g., from the registration generator 322, etc.), and/or boundary data or virtual boundary data (e.g., from the boundary generator 326, etc.). In some embodiments, the planning data includes tracking data (e.g., from the tracking analyzer 324). In some embodiments, the planning data is received from one or more components of the computing system 104, and/or another suitable system or device, for example the robotic system 102 (e.g., via data from the cutting tool 116) and/or a storage device. In an exemplary embodiment, the plan generator 330 is configured to obtain planning data, analyze the planning data, and/or generate one or more surgical, treatment, or operation plans.

In an exemplary embodiment, the plan generator 330 is configured to obtain planning data, analyze the planning data, and generate one or more treatment plans. In some embodiments, the plan generator 330 is configured to create a surgical plan (e.g., a preoperative plan prior to a surgical procedure). The surgical plan may include one or more desired cuts, holes, surfaces, burrs, or other modifications to a patient's anatomy to be made (e.g., using the surgical system 100). The surgical plan may also include one or more boundaries (e.g., a virtual boundary, etc.), restraints, controls, or guides, for example to guide or control a desired movement of one or more components of the surgical system 100 (e.g., the surgical tool 114, the cutting tool 116, etc.). For example, for a total knee arthroscopy procedure, the surgical plan may include the cuts necessary to form, on a femur, a distal surface, a posterior chamfer surface, a posterior surface, an anterior surface, and an anterior chamfer surface in relative orientations and positions suitable to be mated to corresponding surfaces of the prosthetic to be joined to the femur during the surgical procedure, as well as cuts necessary to form, on the tibia, surface(s) suitable to mate to the prosthetic to be joined to the tibia during the surgical procedure. As another example, the surgical plan may include the modifications necessary to create holes in a bone. As another example, in a hip arthroplasty procedure, the surgical plan may include the burr necessary to form one or more surfaces on the acetabular region of the pelvis to receive a cup and, in suitable cases, an implant augment. Accordingly, the plan generator 330 may receive, access, and/or store data facilitate the generation of surgical plans. In some embodiments, the plan generator 330 facilitates intraoperative modifications to the surgical plan.

As described herein, the surgical plan may be developed using pose data, registration data, and/or boundary data or virtual boundary data (e.g., via components of the computing system 104, components of the robotic system 102, via user or operator input, etc.). In some embodiments, the surgical plan is developed using tracking data and/or other suitable data available to the surgical system 100 (e.g., a three-dimensional representation of a patient's anatomy, a virtual bone model, imaging data, model or imaging data stored in a database, one or more models developed by a surgical planning system or software, etc.). In some embodiments, the surgical system 100 is configured to receive input date (e.g., user or operator input data, etc.), for example a user or operator preference (e.g., input) provided via an interaction with the display device 106. In an exemplary embodiment, the plan generator 330 is configured generate the surgical plan, and/or communicate the surgical plan to one or more components of the surgical system 100. For example, the plan generator 330 may generate a surgical plan, and communicate the surgical plan to the robotic system 102, for example to implement the surgical plan (e.g., in accordance with the surgical plan, automatically in accordance with the surgical plan, etc.). In some embodiments, the plan generator 330 is configured to communicate the surgical plan to another system or device (e.g., a computing system, the display device 106, a storage system, etc.), for example for additional analysis, processing, and/or storage.

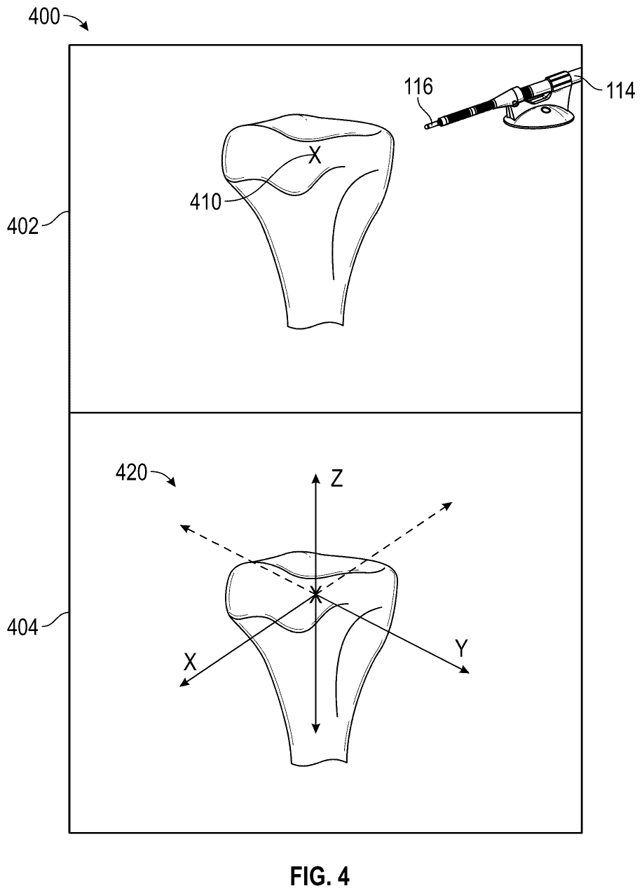

Referring now to FIG. 4, an illustration of an example execution of a process of establishing a coordinate frame is shown, according to an exemplary embodiment. According to an exemplary embodiment, the execution illustrated in FIG. 4 may be implemented using the components of FIGS. 1-3. FIG. 4 shows a storyboard-style illustration 400, where a first frame 402 is shown sequentially followed by a second frame 404.

In an exemplary embodiment, in the first frame 402 one or more objects is/are identified in a space. As shown, a first object 410 may be identified relative to an anatomy of a patient in a space, for example a surgical environment. In an exemplary embodiment, the first object 410 is identified via a pose of the cutting tool 116 of the surgical tool 114. For example, with the anatomy of the patient in the space (e.g., the surgical environment), the cutting tool 116 may be maneuvered to a point in space. The surgical system 100 (e.g., the robotic system 102, the computing system 104, the pose analyzer 320, etc.) may then identify (e.g., determine, mark, etc.) a pose of the first object 410 in space associated with the pose of the cutting tool 116, for example at the three-dimensional position in space of a tip (e.g., tool center point) of the cutting tool 116. The first object 410 may be stored (e.g., with the computing system 104, the robotic system 102, etc.), communicated to another system or device (e.g., the computing system 104, the registration generator 322, etc.), and/or otherwise analyzed or processed. In some embodiments, the first object 410 is identified as a point in space spaced away from the point in space of the cutting tool 116, as discussed herein. In other embodiments, the first object 410 is identified based on a select or particular anatomical feature of a patient (e.g., a lateral condyle of a tibia of a patient, etc.).

In an exemplary embodiment, in the second frame 404 a coordinate frame 420 is generated and/or established in a space. As shown, the coordinate frame 420 may be established relative to the first object 410. For example, the surgical system 100 (e.g., the computing system 104, the registration generator 322, etc.) may be configured to generate the coordinate frame 420 relative to the first object 410, for example with the first object 410 as a center of the coordinate frame 420. In an exemplary embodiment, the coordinate frame 420 is a Cartesian coordinate system, etc.); however, in other embodiments the coordinate frame 420 is another suitable coordinate system (e.g., a cylindrical or spherical coordinate system, homogenous coordinate system, a skewed coordinate system, etc.).

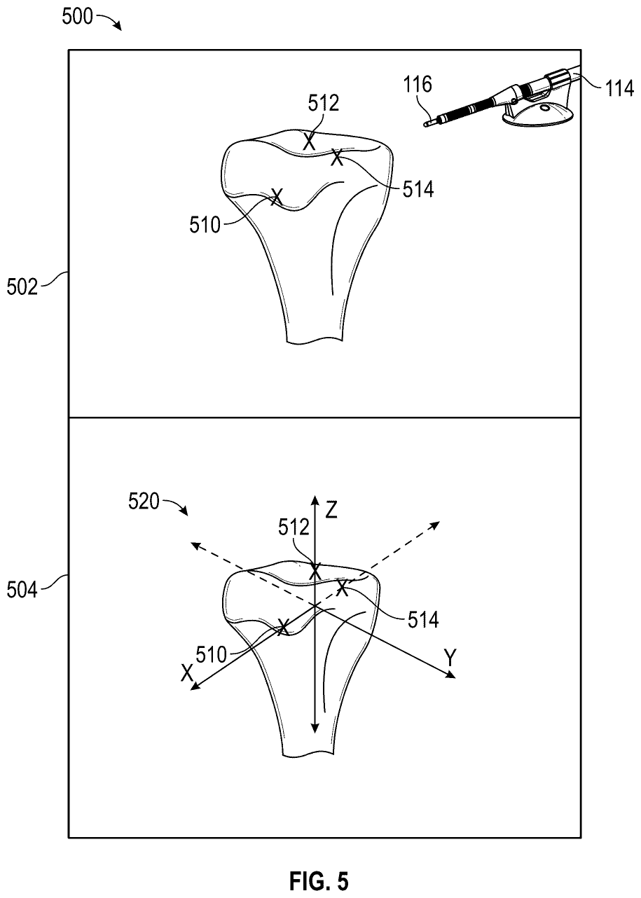

Referring now to FIG. 5, an illustration of an example execution of a process of establishing a coordinate frame is shown, according to an exemplary embodiment. According to an exemplary embodiment, the execution illustrated in FIG. 5 may be implemented using the components of FIGS. 1-3. FIG. 5 shows a storyboard-style illustration 500, where a first frame 502 is shown sequentially followed by a second frame 504.

In an exemplary embodiment, in the first frame 502 one or more objects is/are identified in a space. As shown, a first object 510, a second object 512, and a third object 514 may be identified relative to an anatomy of a patient in a space, for example a surgical environment. In some embodiments, one or more of the identified objects is/are identified based on a select or particular anatomical feature of a patient (e.g., a lateral condyle of a tibia of a patient, etc.). As discussed above, in an exemplary embodiment the first object 510, the second object 512, and/or the third object 514 are identified via a pose of the cutting tool 116 of the surgical tool 114. For example, with the anatomy of the patient in the space (e.g., the surgical environment), the cutting tool 116 may be maneuvered to one or more points in space, for example points at which the cutting tool 116 touches landmarks on the anatomy of the patient. The surgical system 100 (e.g., the robotic system 102, the computing system 104, the pose analyzer 320, etc.) may then identify (e.g., determine, mark, etc.) poses of the first object 510, the second object 512, and/or the third object 514 in space associated with the poses of the cutting tool 116 (e.g., positions of the cutting tool 116 at three points in time as the cutting tool 116 is moved to landmarks on the anatomy of the patient. The first object 510, the second object 512, and/or the third object 514 may be stored (e.g., with the computing system 104, the robotic system 102, etc.), communicated to another system or device (e.g., the computing system 104, the registration generator 322, etc.), and/or otherwise analyzed or processed. In some embodiments, the first object 510, the second object 512, and/or the third object 514 are identified as one or more points in space spaced away from the points in space of the cutting tool 116, as discussed herein. In other embodiments, another suitable number of objects may be identified (e.g., 5, 10, 15, 50, 100, 250, 1,000, etc. objects).

In an exemplary embodiment, in the second frame 504 a coordinate frame 520 is generated and/or established in a space. As shown, the coordinate frame 520 may be established relative to the identified objects (e.g., the first object 510, the second object 512, and/or the third object 514). For example, the surgical system 100 (e.g., the computing system 104, the registration generator 322, etc.) may be configured to generate the coordinate frame 520 relative to the first object 510, the second object 512, and/or the third object 514. In some embodiments, the coordinate frame 520 is generated with a first plane fit to two of the identified points or objects (e.g., the first object 510 and the third object 514), and a second plane fit to one of the identified points or objects (e.g., the second object 512) that intersects with the first plane. In an exemplary embodiment, the coordinate frame 520 is a Cartesian coordinate system, etc.); however, in some embodiments the coordinate frame 520 is another suitable coordinate system (e.g., a cylindrical or spherical coordinate system, homogenous coordinate system, a skewed coordinate system, etc.). In other embodiments, the surgical system 100 (e.g., the computing system 104, the registration generator 322, etc.) is configured to generate or otherwise establish the coordinate frame 520 based on the one or more identified objects (e.g., about an identified object, about a virtual point spaced apart from the identified object, etc.).

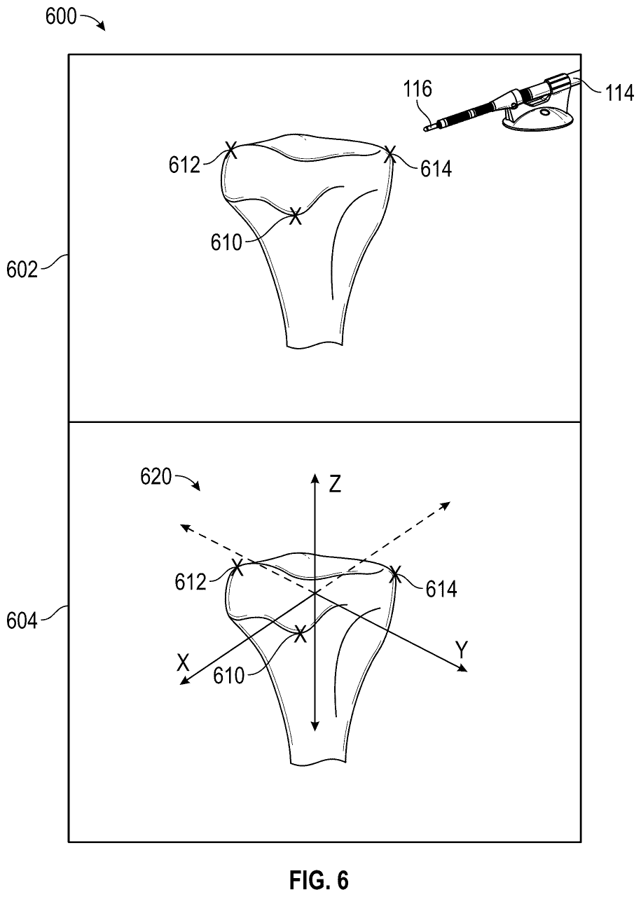

Referring now to FIG. 6, an illustration of an example execution of a process of registering an object is shown, according to an exemplary embodiment. According to an exemplary embodiment, the execution illustrated in FIG. 6 may be implemented using the components of FIGS. 1-3. FIG. 6 shows a storyboard-style illustration 600, where a first frame 602 is shown sequentially followed by a second frame 604.

In an exemplary embodiment, in the first frame 602 one or more objects is/are identified in a space. As shown, a first object 610, a second object 612, and a third object 614 may be identified relative to an anatomy of a patient in a space, for example a surgical environment. In some embodiments, one or more of the identified objects is/are identified based on a select or particular anatomical feature of a patient (e.g., a lateral condyle of a tibia of a patient, etc.). As discussed above, in an exemplary embodiment the first object 610, the second object 612, and/or the third object 614 are identified via a pose of the cutting tool 116 of the surgical tool 114. For example, with the anatomy of the patient in the space (e.g., the surgical environment), the cutting tool 116 may be maneuvered to one or more points in space. The surgical system 100 (e.g., the robotic system 102, the computing system 104, the pose analyzer 320, etc.) may then identify (e.g., determine, mark, etc.) poses of the first object 610, the second object 612, and/or the third object 614 in space associated with the poses of the cutting tool 116. The first object 610, the second object 612, and/or the third object 614 may be stored (e.g., with the computing system 104, the robotic system 102, etc.), communicated to another system or device (e.g., the computing system 104, the registration generator 322, etc.), and/or otherwise analyzed or processed. In some embodiments, the first object 610, the second object 612, and/or the third object 614 are identified as one or more points in space spaced away from the points in space of the cutting tool 116, as discussed herein. In other embodiments, another suitable number of objects may be identified (e.g., 5, 10, 15, 50, 100, 250, 1,000, etc. objects).

In an exemplary embodiment, in the second frame 604 an anatomy of a patient is registered relative to a coordinate frame 620. In some embodiments, the coordinate frame 620 is generated and/or established via the processes described in FIGS. 4-5, for example using the components described in FIGS. 1-3. As shown, the first object 610, the second object 612, and the third object 614 may be registered relative to the coordinate frame 620. In an exemplary embodiment, the registration of the first object 610, the second object 612, and/or the third object 614 relative to the coordinate frame 620 is configured to establish (e.g., determine, identify, generate, etc.) an association between an anatomy of the patient (e.g., associated with the objects 610-614) and a coordinate frame (e.g., the coordinate frame 620). In this regard, the identification of one or more objects (e.g., objects 610-614) and the association of those objects with a coordinate frame (e.g., the coordinate frame 620) allows the surgical system 100 to establish spatial relationships, orientations, and/or configurations between the various components of the surgical system 100 (e.g., between an atomy of a patient and the cutting tool 116, etc.).

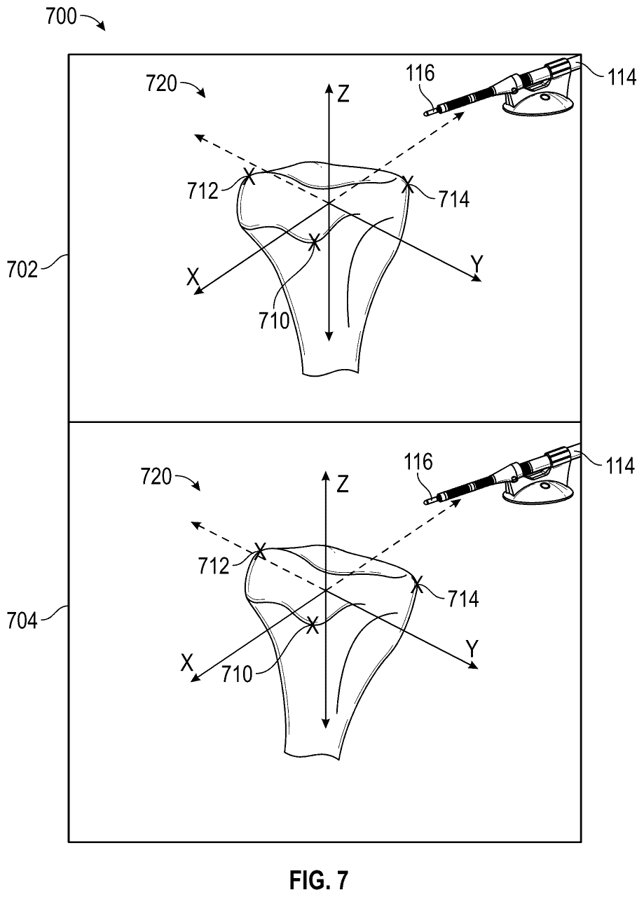

Referring now to FIG. 7, an illustration of an example execution of a process of tracking an object is shown, according to an exemplary embodiment. According to an exemplary embodiment, the execution illustrated in FIG. 7 may be implemented using the components of FIGS. 1-3. FIG. 7 shows a storyboard-style illustration 700, where a first frame 702 is shown sequentially followed by a second frame 704.

In an exemplary embodiment, in the first frame 702 one or more objects is/are identified in a space. As shown, a first object 710, a second object 712, and a third object 714 may be identified relative to an anatomy of a patient in a space, for example a surgical environment. In some embodiments, one or more of the identified objects is/are identified based on a select or particular anatomical feature of a patient (e.g., a lateral condyle of a tibia of a patient, etc.). As discussed above, in an exemplary embodiment the first object 710, the second object 712, and/or the third object 714 are identified via a pose of the cutting tool 116 of the surgical tool 114. The first object 710, the second object 712, and/or the third object 714 may be stored (e.g., with the computing system 104, the robotic system 102, etc.), communicated to another system or device (e.g., the computing system 104, the registration generator 322, a tracking analyzer 324, etc.), and/or otherwise analyzed or processed. For example, and as shown in FIG. 7, the first object 710, the second object 712, and/or the third object 714 may be identified (e.g., registered, associated, determined, etc.) relative to a coordinate frame 720. In some embodiments, the coordinate frame 720 is generated and/or established via the processes described in FIGS. 4-5, and/or the objects 710-714 are registered via the process described in FIG. 6, for example using the components described in FIGS. 1-3. In some embodiments, another suitable number of objects may be identified (e.g., 1, 2, 5, 10, 15, 50, 100, 250, 1,000, etc. objects).

In an exemplary embodiment, in the second frame 704 an anatomy of a patient is tracked or monitored relative to the coordinate frame 720. As indicated above, information (e.g., pose data, etc.) relating to the first object 710, the second object 712, and/or the third object 714 may be communicated to and/or analyzed by one or more components of the surgical system 100 (e.g., the tracking analyzer 324, etc.). For example, pose data associated with the first object 710, the second object 712, and/or the third object 714 may be communicated and/or analyzed in sequence (e.g., over a period of time, over a surgical procedure, etc.). For example, a first set of pose data associated with the first object 710, the second object 712, and/or the third object 714 may be determined at a first point in time (e.g., during a registration or calibration phase, etc.), for example as shown in the first frame 702. Subsequently, a second set of pose data associated with the first object 710, the second object 712, and/or the third object 714 may be determined at a second point in time (e.g., during an interoperative workflow phase, during a bone cutting phase, during a preparation phase, etc.), as shown in the second frame 704. The second set of pose data may be determined via a pose of the cutting tool 116, for example by positioning and/or moving the cutting tool 116 back to the first object 710, the second object 712, and/or the third object 714 (e.g., retuning the cutting tool 116 to a checkpoint associated with the objects 710-714, etc.). In an exemplary embodiment, the first set of pose data (e.g., at a first point in time, etc.) is compared to the second set of pose data (e.g., at a second point in time, etc.), for example to track and/or monitor movement of the first object 710, the second object 712, and/or the third object 714 (e.g., within the coordinate frame) over time. In this regard, respective sets of pose data associated with the first object 710, the second object 712, and/or the third object 714 may be analyzed over time (e.g., preceding pose data, subsequent pose data, etc.) and/or relative to the coordinate frame 720, in order to track and/or monitored movement of an anatomy of a patient.

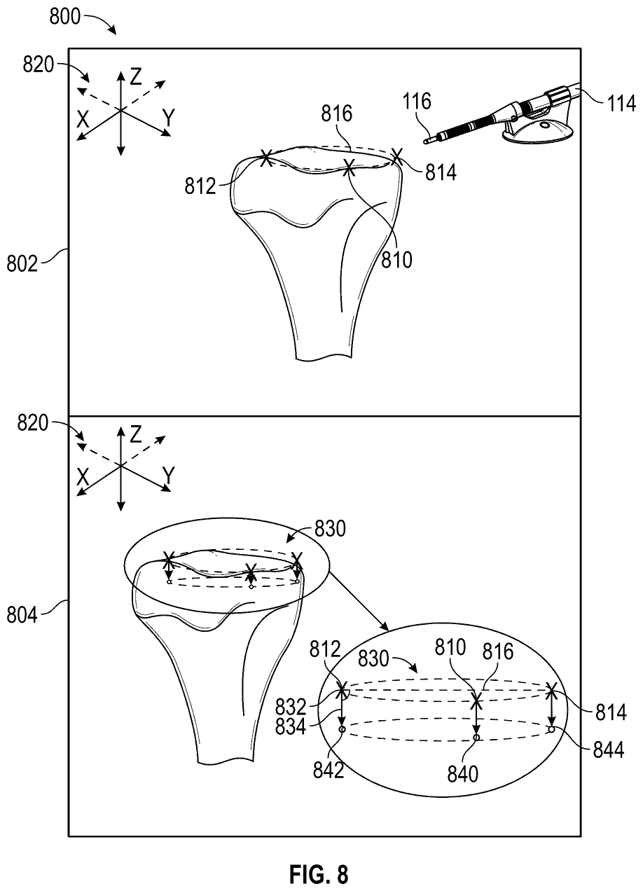

Referring now to FIG. 8, an illustration of an example execution of a process of generating a virtual boundary is shown, according to an exemplary embodiment. According to an exemplary embodiment, the execution illustrated in FIG. 8 may be implemented using the components of FIGS. 1-3. FIG. 8 shows a storyboard-style illustration 800, where a first frame 802 is shown sequentially followed by a second frame 804.

In an exemplary embodiment, in the first frame 802 one or more objects is/are identified in a space. As shown, the one or more objects may include three objects; however, in some embodiments the one or more objects include another suitable number of objects (e.g., 2, 5, 10, 25, 50, 100, etc. objects). As shown in the first frame 802, a first object 810, a second object 812, and a third object 814 may be identified relative to an anatomy of a patient in a space, for example a surgical environment. As discussed herein, the first object 810, the second object 812, and/or the third object 814 may be an identified point in space (e.g., a (X, Y, Z) coordinate point in a coordinate frame, etc.), which may be stored, analyzed, processed, and/or manipulated (e.g., via one or more components of the surgical system 100). In some embodiments, one or more of the identified objects is/are identified based on an association with an anatomical feature of a patient (e.g., a lateral condyle of a tibia of a patient, etc.). In some embodiments, one or more of the identified objects is/are identified based on a tracing of a surface of an anatomy of a patient. For example, the first object 810, the second object 812, and/or the third object 814 may be identified by tracing (e.g., via a pose of the cutting tool 116 of the surgical tool 114) a surface of an anatomy of a patient (e.g., a surface of a tibia of a patient, a skin of a patient on a joint of the patient, etc.). As shown in the first frame 802, the surgical system 100 (e.g., the robotic system 102, the computing system 104, the boundary generator 326, etc.) may also be configured to establish (e.g., fit, etc.) a plane that includes or is best-fit to the identified objects. For example, the surgical system 100 (e.g., the boundary generator 326, etc.) may establish a first plane 816, which includes the first object 810, the second object 812, and the third object 814. The first plane 816 (e.g., including the first object 810, the second object 812, and/or the third object 814) may be defined in the coordinate system of a coordinate frame 820 (e.g., a coordinate frame established and/or generated via the processes described in FIGS. 4-5, for example using the components described in FIGS. 1-3, etc.).

In an exemplary embodiment, in the second frame 804 a virtual boundary 830 is established (e.g., generated, determined, etc.) in a space (e.g., the surgical environment). In some embodiments, the virtual boundary 830 is or includes a projection based on the identified objects (e.g., the first object 810, the second object 812, the third object 814, etc.). For example, the surgical system 100 (e.g., the robotic system 102, the computing system 104, the boundary generator 326, etc.) may be configured to determine an orientation (e.g., shown as angle 832) relative to the first plane 816, and project a virtual boundary a distance (e.g., shown as distance 834) from the first plane 816. For example, the surgical system 100 (e.g., the boundary generator 326) may be configured to determine the angle 832 (e.g., a 90-degree angle, etc.) relative to the first plane 816, and project a virtual boundary the distance 834 (e.g., 1 millimeter, etc.) from the first plane 816 in the direction of the angle 832. The virtual boundary may also include a plurality of virtual vertices, for example associated with each of the identified objects (e.g., spaced apart from the identified objects by the distance 834, oriented relative to the identified objects by the angle 832, etc.). For example, the virtual boundary may include a first virtual vertex 840 associated with the first object 810 (e.g., spaced the distance 834 apart from the first object 810 at the angle 832, etc.), a second virtual vertex 842 associated with the second object 812, and/or a third virtual vertex 844 associated with the third object 814). In some embodiments, the surgical system 100 (e.g., the robotic system 102, the computing system 104, the boundary generator 326, etc.) is also be configured to establish (e.g., fit, etc.) a plane that includes the virtual vertices. For example, the surgical system 100 (e.g., the boundary generator 326) may be configured to generate a virtual boundary that establishes a virtual plane 846, which includes the first virtual vertex 840, the second virtual vertex 842, and/or the third virtual vertex 844.

As described herein, in some embodiments the distance 834 is associated with a cutting depth (e.g., a selected cutting depth, a user-selected cutting depth, etc.). In some embodiments, the angle 832 and/or the distance 834 are angles and/or distances, for example established via a setting or configuration. In some embodiments, the angle 832 and/or the distance 834 are selected or determined via an input (e.g., a user or operator input, etc.), a program or application, and/or via another suitable system or device. It should be understood that the angle 832 and/or the distance 834 may be any suitable orientation (e.g., e.g., 5, 10, 15, 30, 45, 60, 75, 80, 90, 100, etc. degrees relative to the plane) and/or distance (e.g., 0.01, 0.5, 1, 1.5, 2, 2.5, 5, 10, etc. millimeters or centimeters, etc.).

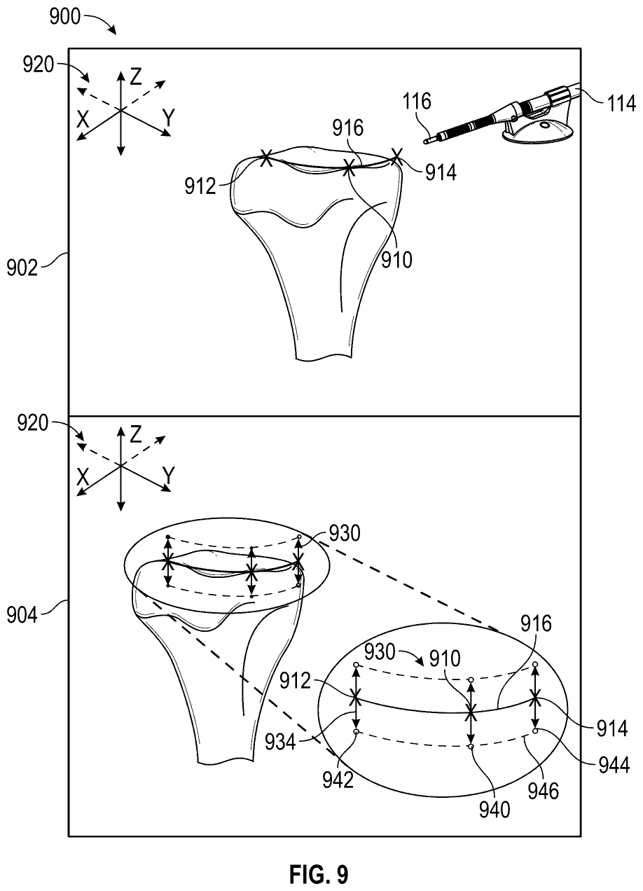

Referring now to FIG. 9, an illustration of an example execution of a process of generating a virtual boundary is shown, according to an exemplary embodiment. According to an exemplary embodiment, the execution illustrated in FIG. 9 may be implemented using the components of FIGS. 1-3. FIG. 9 shows a storyboard-style illustration 900, where a first frame 902 is shown sequentially followed by a second frame 904.

In an exemplary embodiment, in the first frame 902 one or more objects is/are identified in a space. For example, in the first frame 902 the cutting tool 116 may be moved to (e.g., touched, contacted to, positioned at, etc.) a plurality of points on an anatomy of a patient, for example a user-selected number of points. In an exemplary embodiment, the cutting tool 116 is moved to at least three points on one or more bones of a patient. As the cutting tool 116 is moved to the points, the points can be collected (e.g., determined or identified via tracking or pose data indicative of a pose of the cutting tool 116), and/or stored as objects, shown as a first object 912, a second object 914, and/or a third object 914. In this regard, the objects 910-914 may represent stored positions of the cutting tool 116 at different locations at the anatomy (e.g., bones, etc.) of a patient. In some embodiments, the objects 910-914 may be identified (e.g., determined, collected, etc.), for example in response to a user engaging with and/or providing an input to a component of the surgical system 100. For example, the objects 910-914 may be collected in response to a user engaging the button 220, for providing another input indicating that a pose should be collected (e.g., a depression of a foot pedal, engagement with the display device 106, a voice command, etc.).

As shown in the first frame 902, the surgical system 100 (e.g., the robotic system 102, the computing system 104, the boundary generator 326, etc.) may also be configured to establish (e.g., fit, etc.) a line that includes the identified objects. For example, the surgical system 100 (e.g., the boundary generator 326, etc.) may establish a first line 916, which includes the first object 910, the second object 912, and the third object 914. The first line 916 (e.g., including the first object 910, the second object 912, and/or the third object 914) may further be established and/or associated with a coordinate frame 920 (e.g., a coordinate frame established and/or generated via the processes described in FIGS. 4-5, for example using the components described in FIGS. 1-3, etc.).

In an exemplary embodiment, in the second frame 904 a virtual boundary 930 is established (e.g., generated, determined, etc.) in a space (e.g., the surgical environment). In some embodiments, the virtual boundary 930 is or includes a projection based on the identified objects (e.g., the first object 910, the second object 912, the third object 914, etc.). For example, the surgical system 100 (e.g., the robotic system 102, the computing system 104, the boundary generator 326, etc.) may be configured to determine an orientation (e.g., an angle 932) relative to the first line 916, and/or project a virtual boundary a distance (e.g., shown as distance 834) from the first line 916. For example, the surgical system 100 (e.g., the boundary generator 326) may be configured to determine an orientation of the first line 916 in the coordinate frame 920 (e.g., the angle 932, etc.), and project a virtual boundary the distance 834 (e.g., 1 millimeter, etc.) from the first line 916 in the direction of the orientation (e.g., in a vertical direction aligned with a Z-axis of the coordinate frame 920). As shown in the second frame 904, the virtual boundary may extend the distance 834 from the first line 916 in a plurality of directions (e.g., in vertically opposing directions aligned with a Z-axis of the coordinate frame 920, etc.). In some embodiments, the virtual boundary also includes one or more virtual vertices. For example, the virtual boundary may include a first virtual vertex 940 associated with the first object 910 (e.g., spaced the distance 934 apart from the first object 910, etc.), a second virtual vertex 942 associated with the second object 912, and/or a third virtual vertex 944 associated with the third object 914). Further, as described herein the surgical system 100 (e.g., the boundary generator 326) may be configured to generate a virtual boundary that establishes one or more virtual lines, shown as a virtual line 946, for example which includes the first virtual vertex 940, the second virtual vertex 942, and/or the third virtual vertex 944.

As described herein, in some embodiments the distance 934 is associated with a cutting depth, which can be established via predetermined programs, applications, and/or other suitable systems or devices. In some embodiments, the distance 934 is determined via an input (e.g., a user or operator input, etc.). It should be understood that the orientation of the virtual boundary and/or the distance 834 may be any suitable orientation (e.g., e.g., 5, 10, 15, 30, 45, 60, 75, 80, 90, 100, etc. degrees relative to an axis or plane of the coordinate frame 920, etc.) and/or distance (e.g., 0.01, 0.5, 1, 1.5, 2, 2.5, 5, 10, etc. millimeters or centimeters, etc.).

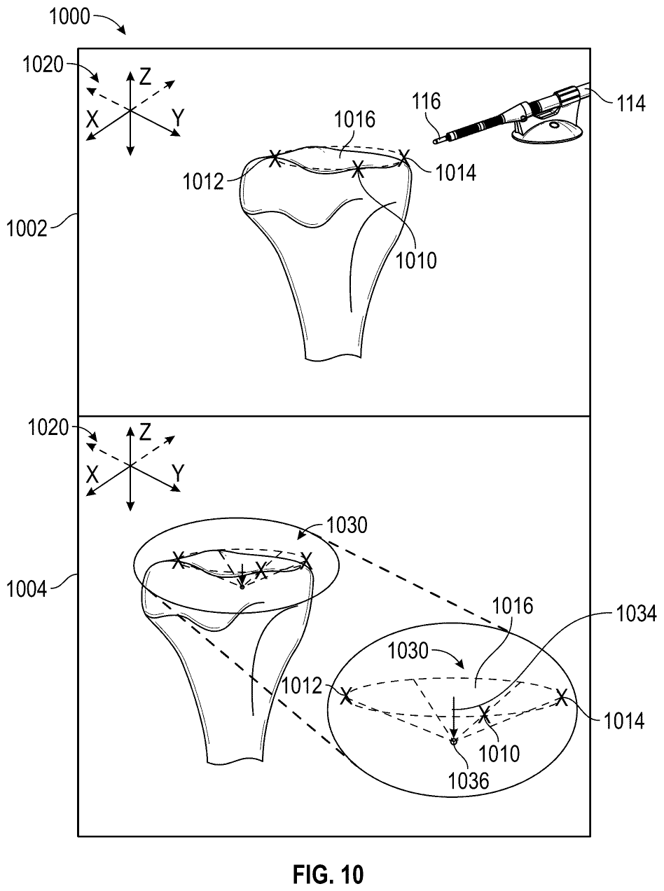

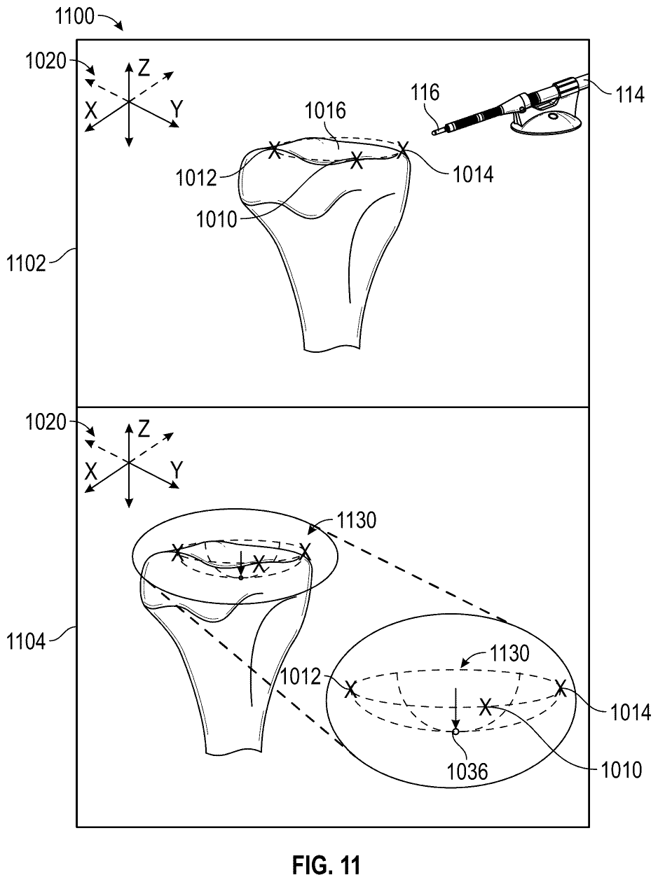

Referring now to FIGS. 10-11, illustrations of an example execution of processes of generating virtual boundaries are shown, according to exemplary embodiments. According to an exemplary embodiment, the execution illustrated in FIGS. 10-11 may be implemented using the components of FIGS. 1-3. FIG. 10 shows a storyboard-style illustration 1000, where a first frame 1002 is shown sequentially followed by a second frame 1004. FIG. 11 shows a storyboard-style illustration 1100, where a first frame 1102 is shown sequentially followed by a second frame 1104.

In an exemplary embodiment, in a first frame (e.g., the first frame 1002, the first frame 1102, etc.) one or more objects is/are identified in a space. As shown, the one or more objects may include three objects; however, in some embodiments the one or more objects include another suitable number of objects (e.g., 2, 5, 10, 25, 50, 100, etc. objects). As shown, a first object 1010, a second object 1012, and a third object 1014 may be identified relative to an anatomy of a patient in a space, for example a surgical environment. In some embodiments, one or more of the identified objects is/are identified based on a select or particular anatomical feature of a patient (e.g., a lateral condyle of a tibia of a patient, etc.). In some embodiments, one or more of the identified objects is/are identified based on a tracing of a surface of an anatomy of a patient. For example, the first object 1010, the second object 1012, and/or the third object 1014 may be identified by tracing (e.g., via a pose of the cutting tool 116 of the surgical tool 114) a surface of an anatomy of a patient (e.g., a surface of a tibia of a patient, a skin of a patient on a joint of the patient, etc.).

As shown in the first frame (e.g., the first frame 1002, the first frame 1102, etc.), in some embodiments the surgical system 100 (e.g., the robotic system 102, the computing system 104, the boundary generator 326, etc.) is also configured to establish (e.g., fit, etc.) a plane that includes the identified objects. For example, the surgical system 100 (e.g., the boundary generator 326, etc.) may establish a first plane 1016, which includes the first object 1010, the second object 1012, and/or the third object 1014. The first plane 1016 (e.g., including the first object 1010, the second object 1012, and/or the third object 1014) may further be established and/or associated with a coordinate frame 1020 (e.g., a coordinate frame established and/or generated via the processes described in FIGS. 4-5, for example using the components described in FIGS. 1-3, etc.).

In an exemplary embodiment, in the second frame a virtual boundary is established (e.g., generated, determined, etc.) in a space (e.g., the surgical environment). In an exemplary embodiment, the virtual boundary is or includes a projection based on the identified objects (e.g., the first object 1010, the second object 1012, the third object 1014, etc.). Further, the virtual boundary may be or include a predefined shape or configuration. For example, as shown in the second frame 1004, a virtual boundary 1030 may be a cone or pyramid shape. Further, as shown in the second frame 1104, a virtual boundary 1130 may be a hemisphere shape.

As discussed above, in an exemplary embodiment the surgical system 100 (e.g., the robotic system 102, the computing system 104, the boundary generator 326, etc.) is configured to establish a virtual boundary (e.g., the virtual boundary 1030, the virtual boundary 1130, etc.) using one or more of the identified objects (e.g., the first object 1010, the second object 1012, and/or the third object 1014, etc.). For example, the surgical system 100 may define a base of the virtual boundary to include a plurality of the identified objects (e.g., in the first plane 1016, etc.), and a virtual projection a distance (e.g., shown as distance 1034, etc.) from the base as an apex 1036 of the virtual boundary. As shown in the second frame 1004, the surgical system 100 may be configured to generate the virtual boundary 1030 (e.g., connecting the base and the apex 1036), such that the virtual boundary 1030 is a prism shape. As shown in the second frame 1104, the surgical system 100 may also be configured to generate the virtual boundary 1130 (e.g., connecting the base and the apex 1036), such that the virtual boundary 1130 is a hemisphere shape.

It should be understood that in some embodiments, the virtual boundary (e.g., the virtual boundary 1030, the virtual boundary 1130, etc.) is defined such that a portion of the identified objects define a base of the virtual boundary, and/or one of the identified objects defines an apex of the virtual boundary. In other embodiments, the surgical system 100 is configured to use the identified objects (e.g., the first object 1010, the second object 1012, and/or the third object 1014, etc.) to generate a virtual boundary that is another suitable shape and/or configuration (e.g., sphere, cylinder, cube, triangular prism, square, triangle, circle, etc.). As such, it is contemplated that the surgical system 100 may uses any combination of the identified objects and/or projections (e.g., a virtual projection, etc.) to define one or more surfaces, points (e.g., center point, point of rotation, etc.), planes, axis, and/or edges of the shapes and/or configurations of a virtual boundary (e.g., a base, a side edge, an apex, a portion of a circumference, etc.). Further, it should be understood that while the virtual boundary (e.g., the virtual boundary 1030, the virtual boundary 1130, etc.) is described as being oriented relative to the coordinate frame 1020 and/or the distance 1034 is described as being associated with a cutting depth, in other embodiments the virtual boundary is otherwise oriented and/or the distance 1034 is otherwise determined and/or defined.

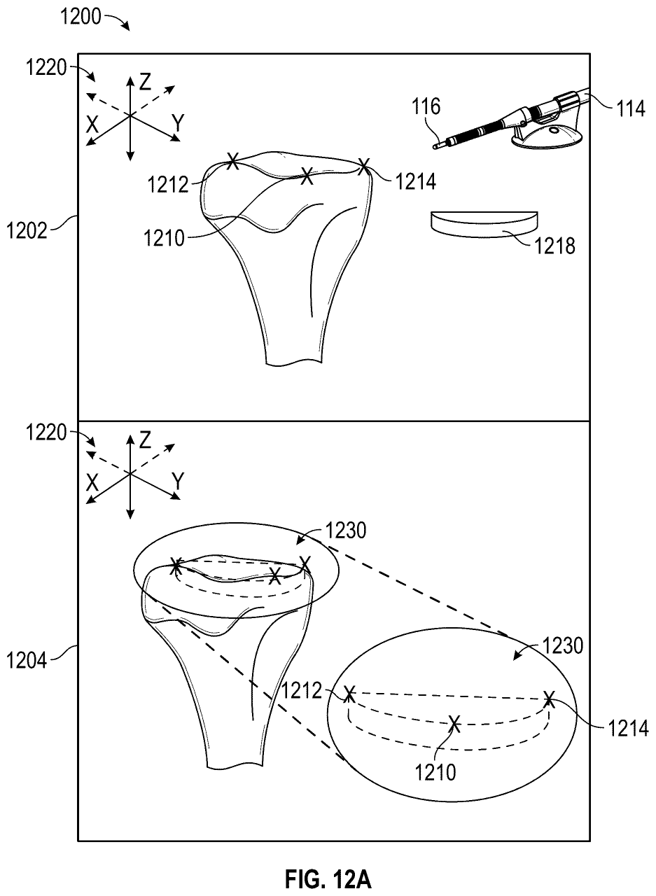

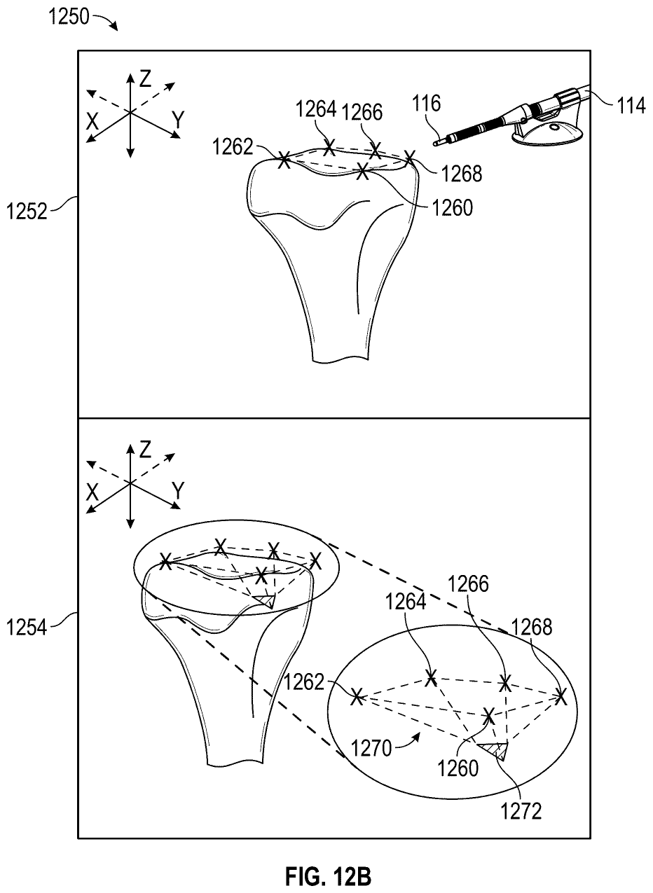

Referring now to FIG. 12A, an illustration of an example execution of a process of generating a virtual boundary is shown, according to an exemplary embodiment. According to an exemplary embodiment, the execution illustrated in FIG. 12A may be implemented using the components of FIGS. 1-3. FIG. 12A shows a storyboard-style illustration 1200, where a first frame 1202 is shown sequentially followed by a second frame 1204.

In an exemplary embodiment, in the first frame 1202 one or more objects is/are identified in a space. As shown, the one or more objects may include three objects; however, in some embodiments the one or more objects include another suitable number of objects (e.g., 2, 5, 10, 25, 50, 100, etc. objects). As shown, a first object 1210, a second object 1212, and a third object 1214 may be identified relative to an anatomy of a patient in a space, for example a surgical environment. In some embodiments, one or more of the identified objects is/are identified based on a select or particular anatomical feature of a patient (e.g., a lateral condyle of a tibia of a patient, etc.), for example based on a tracing of a surface of an anatomy of a patient (e.g., via a pose of the cutting tool 116 of the surgical tool 114). In an exemplary embodiment, using the identified objects (e.g., the first object 1210, the second object 1212, and/or the third object 1214, etc.), the surgical system 100 (e.g., the robotic system 102, the computing system 104, the boundary generator 326, etc.) is configured to identify an implant 1218. For example, using the identified objects, the surgical system 100 may identify the implant 1218 via one or more virtual implant models stored in the surgical system 100 and/or another storage device (e.g., a storage database, etc.).

In an exemplary embodiment, in the second frame 1204 a virtual boundary 1230 is established (e.g., generated, determined, etc.) in a space (e.g., the surgical environment). In an exemplary embodiment, the virtual boundary 1230 is or includes a projection based on the identified objects (e.g., the first object 1210, the second object 1212, the third object 1214, etc.). For example, the virtual boundary 1230 may be or include a projection based on the implant 1218. In this regard, the virtual boundary 1230 may be a projection that includes virtual boundaries associated with the implant 1218. In some embodiments, the virtual boundary 1230 includes virtual boundaries associated with other spaces, features, and/or components (e.g., bone screws, bone cement, etc.).

Referring now to FIG. 12B, an illustration of an example execution of a process of generating a virtual boundary is shown, according to an exemplary embodiment. According to an exemplary embodiment, the execution illustrated in FIG. 12B may be implemented using the components of FIGS. 1-3. FIG. 12B shows a storyboard-style illustration 1200, where a first frame 1252 is shown sequentially followed by a second frame 1254.

In an exemplary embodiment, in the first frame 1202 one or more objects is/are identified in a space. As shown, the one or more objects can include any user-selected number of objects (points, etc.), (e.g., 2, 5, 10, 25, 50, 100, etc. objects). As shown, a first object 1260, a second object 1262, a third object 1264, a fourth object 1266, and a fifth object 1268 are identified relative to an anatomy of a patient in a space, for example a surgical environment. The objects are selected by touching the cutting tool 116 of the surgical tool 114 to the positions represented by the objects 1260, 1262, 1264, 1266, 1268, for example by tracing of a surface of an anatomy of the cutting tool 116 of the surgical tool 114 while its pose is monitored (e.g., via encoders of joints of a robotic arm, via optical tracking, etc.). To provide the first frame 1252, the cutting tool 116 can be touched to any number of points that a user wishes to capture and use for generation of a virtual boundary, for example arranged in any arbitrary shape as created by user input.

In an exemplary embodiment, in the second frame 1254 a virtual boundary 1270 is established (e.g., generated, determined, etc.) in a space (e.g., the surgical environment). In an exemplary embodiment, the virtual boundary 1270 is defined by a projection from the objects 1260, 1262, 1264, 1266, 1268 to a selected shape (shown as triangle 1272) at a cutting depth from the objects 1260, 1262, 1264, 1266, 1268. As shown, a boundary can be defined by drawing line segments (e.g., linear segments, curves) between neighboring objects of the objects 1260, 1262, 1264, 1266, 1268 to define a base of a prism, and connecting the objects 1260, 1262, 1264, 1266, 1268 to vertices of a selected shape shown as triangle 1272) at a cutting depth from the base to generate sides of the prism. The generated prism or a portion thereof can then be used as the virtual boundary 1270.

The example shown in FIG. 12B is illustrative and illustrates that any number of points (objects) can be selected in any arbitrary shape or arrangement as input by a user, and used to generate a virtual boundary according to the teachings herein. Furthermore, the selected shape (e.g., user-selected shape) can be a user-selected basic or regular shape, an arbitrary polygon defined by a user, a circle, a line, a shape corresponding to a virtual model of an implant component, etc., in various embodiments (e.g., based on user selections and settings). Accordingly, the teachings herein provide for wide customizability to accommodate various clinical scenarios which may face surgeon users of the systems and methods taught herein.

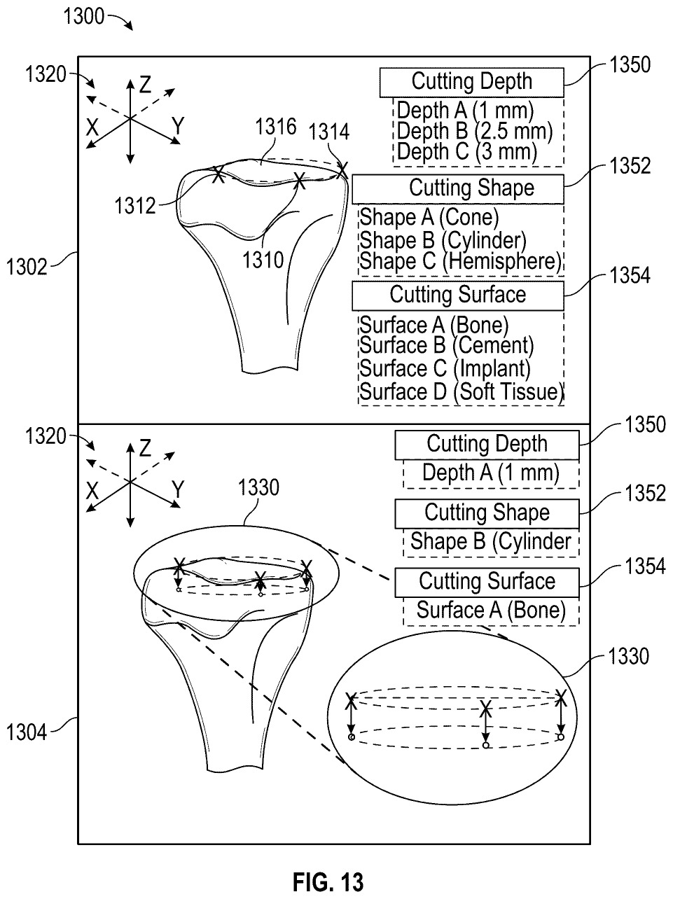

Referring now to FIG. 13, an illustration of an example interfaces associated with execution of a process of generating a virtual boundary is shown, according to an exemplary embodiment. According to an exemplary embodiment, the execution illustrated in FIG. 13 may be implemented using the components of FIGS. 1-3. FIG. 13 shows a storyboard-style illustration 1300, where a first interface 1302 is shown sequentially followed by a second interface 1304.

In an exemplary embodiment, in the first interface 1302 one or more objects is/are illustrated in a space. As shown, a first object 1310, a second object 1312, and a third object 1314 are shown relative to an anatomy of a patient in a space, for example a surgical environment. Further, a first plane 1316 is shown, which may include the first object 1310, the second object 1312, and/or the third object 1314. The first interface 1302 may further include a coordinate frame 1320, which may provide a relative orientation and/or configuration of one or more features illustrated on the first interface 1302 (e.g., the first object 1310, the second object 1312, the third object 1314, the first plane 1316, etc.).

According to an exemplary embodiment, the first interface 1302 further includes one or more selectable icons (e.g., buttons, toggles, etc.). As shown, the first interface 1302 includes a first selectable icon 1350, a second selectable icon 1352, and a third selectable icon 1354. The selectable icons 1350-1354 may be associated with one or more of the features and/or processes described herein. For example, the first selectable icon 1350 may be associated with a distance, for example a cutting depth. In response to a selection of the first selectable icon 1350, the first interface 1302 may display a plurality of options associated with the first selectable icon 1350 (e.g., a first distance, a second distance, a third distance, a customizable distance, etc.). The second selectable icon 1352 may be associated with a shape or configuration, for example a shape or configuration associated with a virtual boundary. In response to a selection of the second selectable icon 1352, the first interface 1302 may display a plurality of options associated with the second selectable icon 1352 (e.g., a first shape, a second shape, a shape of an implant, a plane or axis, a customizable or uploaded shape, etc. associated with a virtual boundary, etc.). The third selectable icon 1354 may be associated with a surface, material, or substance (e.g., a soft tissue, etc.), for example a material of a component to be cut or altered. In response to a selection of the third selectable icon 1354, the first interface 1302 may display a plurality of options associated with the third selectable icon 1354 (e.g., a first material, a second material, a customizable material, etc. associated with a component to be cut or altered).

In an exemplary embodiment, in the second interface 1304 a virtual boundary 1330 is illustrated in space. For example, the virtual boundary 1330 may be illustrated relative to an anatomy of a patient. According to an exemplary embodiment, the virtual boundary 1330 is provided based on one or more inputs (e.g., a user input, an operator input, a predetermined setting or input, etc.). For example, a user or operate may interact with the selectable icons 1350-1354, for example to provide the surgical system 100 with one or more inputs (e.g., associated with the selectable icons 1350-1354). Based on one or more interactions with the selectable icons 1350-1354, the surgical system 100 may be configured to generate a virtual boundary (e.g., in accordance with, using, etc. the inputs), and display the virtual boundary as the virtual boundary 1330 in the second interface 1304.

It should be understood that while the interfaces 1302-1304 are described as having certain selectable icons, it is contemplated that in other embodiments the interfaces 1302-1304 include additional and/or different components (e.g., icons, indicators, etc.). For example, in some embodiments the second interface 1304 may include one or more messages or alerts associated with the virtual boundary 1330 (e.g., a message including instructions for implementing a procedure, an alert identifying one or more boundaries that exceed a threshold, for example relating to cutting depth or material removal, etc.). In other embodiments, the interfaces 1302-1304 include components are associated with other suitable features, components, and/or processes described herein.

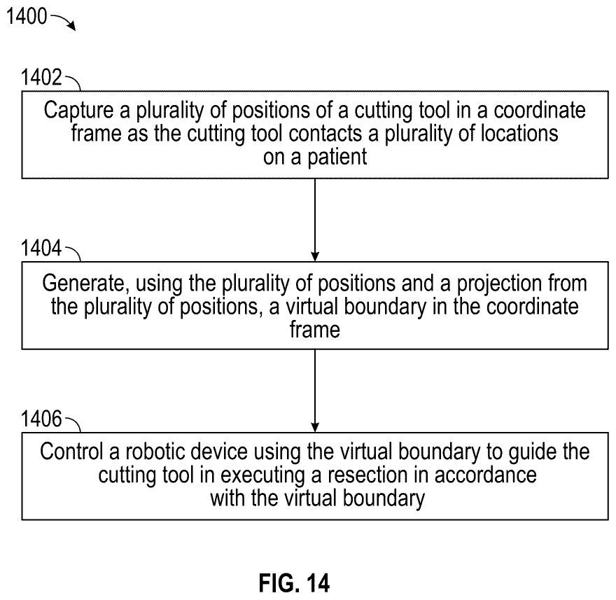

Referring now to FIG. 14, a flow diagram of a process 1400 that can be executed by the surgical system 100 of FIGS. 1-3 is shown, according to an exemplary embodiment. In an exemplary embodiment, process 1400 may be implemented to provide a virtual boundary. Process 1400 may be implemented and/or adapted to facilitate various surgical procedures (e.g., a joint replacement surgery, etc.).

At step 1402, a plurality of positions of an object are obtained or identified. According to an exemplary embodiment, a plurality of positions of a cutting tool (e.g., the cutting tool 116) are obtained. The plurality of positions may be obtained relative to a coordinate frame. In some embodiments, the coordinate frame is established and/or generated via the processes described in FIGS. 4-5. According to an exemplary embodiment, the plurality of positions are identified as the object contacts a plurality of locations on a patient. For example, the plurality of positions may be identified as the cutting tool 116 contacts a plurality of locations on an anatomy of a patient (e.g., traces a surface of an anatomy of a patient, traces a surface of a bone of a patient, etc.). The plurality of positions may include at least three positions; however, in other embodiments the plurality of positions includes another suitable number of positions. In some embodiments, the plurality of positions are captured continuously, for example as the cutting tool 116 traces a portion of an anatomy of a patient (e.g., a bone, a joint, etc.).

At step 1404, a virtual boundary is generated. According to an exemplary embodiment, a virtual boundary is generated using the plurality of positions (e.g., the plurality of positions of the cutting tool 116) at the plurality of locations on the anatomy of the patient. In an exemplary embodiment, the virtual boundary is or includes a projection. For example, the virtual boundary may be or include a plurality of projections from the plurality of positions on the anatomy of the patient. In some embodiments, the plurality of projections are spaced a distance (e.g., a select distance, a user-selected distance, etc.) from the plurality of positions on the anatomy of the patient. For example, the plurality of projections may be spaced a cutting distance (e.g., a select cutting distance, a use-selected distance defined by an input, etc.). As described herein, the virtual boundary may be or include one or more shapes or configurations (e.g., a cone, a pyramid, a hemisphere, an implant, etc.), for example defined by and/or generated using the plurality of positions on the anatomy of the patient. In some embodiments, the virtual boundary is generated relative to the coordinate frame. According to an exemplary embodiment, the virtual boundary establishes and/or provides a guide or restraint (e.g., a boundary, a limit, a threshold, etc.), for example to control or guide manipulation of a tool or instrument in a space (e.g., a surgical field, a surgical environment, the coordinate frame, etc.).

In some embodiments, the projection is generated by fitting a plane to the plurality of positions (or a portion thereof). Further, generating the projection may include determining a direction normal to the plane (e.g., into a patient, etc.), and/or defining a plurality of vertices spaced apart from the plurality of positions (e.g., a distance of the selected cutting depth, etc.). In some embodiments, the virtual boundary intersects the plurality of vertices and/or the plurality of positions. In some embodiments, the projection and/or the virtual boundary is generated using the plurality of positions and/or a distance (e.g., a predefined cutting depth, etc.). For example, the projection may be generated as a cone, such that the plurality of positions (or a portion thereof) define a base of the cone, and an apex of the cone is at a distance (e.g., a cutting depth) from the base. In some embodiments, the projection is a hemisphere, for example defined by plurality of positions (e.g., a base of the hemisphere, etc.), and projecting from the base (e.g., into a patient from the base, etc.). In other embodiments, the projection and/or the virtual boundary is generated based on an implant selected based on the plurality of positions, such that the virtual boundary (e.g., selected implant, etc.) matches or coincides with the plurality of positions on and/or at the anatomy of the patient. In some embodiments, a plurality of projections and/or virtual boundaries are generated. The plurality of projections and/or virtual boundaries may be associated with one or more force feedback and/or force feedback characteristics. For example, a first virtual boundary may provide force feedback having a first characteristic (e.g., responsive to the cutting tool interacting with the first virtual boundary, etc.), and/or a second virtual boundary may provide force feedback having a second characteristic (e.g., responsive to the cutting tool interacting with the second virtual boundary, etc.), where the first force feedback may feel different (e.g., in frequency, strength, etc.) to a user compared to the second force feedback.

At step 1406, a robotic device is controlled in accordance with the virtual boundary. According to an exemplary embodiment, the robotic device is controlled using the virtual boundary, for example to guide the cutting tool. The robotic device may be controlled using the virtual boundary to guide the cutting tool is executing a resection, for example in accordance with the virtual boundary.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. For example, the process of establishing a coordinate frame of the exemplary embodiment described in at least FIG. 5 may be incorporated in the process of providing a virtual boundary of the exemplary embodiment described in at least FIG. 14. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A surgical system, comprising:
a robot;
a cutting tool coupled to the robot; and
a computing system programmed to:
capture a plurality of positions of the cutting tool in a coordinate frame as the cutting tool contacts a plurality of locations on a patient;
generate, using the plurality of positions and a projection from the plurality of positions to a selected cut depth, a virtual boundary in the coordinate frame; and
control the robot using the virtual boundary to guide the cutting tool in executing a resection in accordance with the virtual boundary.

2. The surgical system of claim 1, wherein the plurality of captured positions comprises a user-selectable number of captured positions.

3. The surgical system of claim 1, wherein the plurality of captured positions are captured continuously as the cutting tool traces a portion of a joint of the patient.

4. The surgical system of claim 1, wherein the computing system is programmed to generate the projection from the plurality of positions to the selected cut depth by:
fitting a plane to the plurality of positions;
determining a direction normal to the plane and into the patient; and
defining a plurality of vertices spaced apart from the plurality of positions in the direction by the selected cut depth,
wherein the virtual boundary intersects the plurality of vertices and the plurality of positions.

5. The surgical system of claim 1, wherein the computing system is programmed to generate the projection from the plurality of positions to the selected cut depth by projecting a cone from the plurality of positions such that the plurality of positions define a base of the cone and such that an apex of the cone is at the selected cut depth from the base.

6. The surgical system of claim 1, wherein the virtual boundary is a hemisphere defined by the plurality of captured positions of the cutting tool and projecting into the patient from the plurality of captured positions of the cutting tool.

7. The surgical system of claim 1, wherein the computing system is programed to generate, using the plurality of positions and the projection from the plurality of positions to the selected cut depth, the virtual boundary in the coordinate frame further based on a surface geometry of a selected implant.

8. The surgical system of claim 1, wherein the virtual boundary is a first virtual boundary of a plurality of virtual boundaries, and wherein the computing system is further programed to:
capture a second plurality of positions of the cutting tool in the coordinate frame as the cutting tool contacts a second plurality of portions of locations on the patient; and
generate, using the second plurality of positions, a second virtual boundary in the coordinate frame.

9. The surgical system of claim 8, wherein the computing system is programmed to control the robot further using the second virtual boundary by providing first force feedback having a first characteristic responsive to interactions between the cutting tool and the first virtual boundary and second force feedback having a second characteristic responsive to interactions between the cutting tool and the second virtual boundary, wherein the first characteristic differs from the second characteristics such that the first force feedback feels different to a user than the second force feedback.

10. The surgical system of claim 1, wherein the computing system is further programed to:
capture at least one registration position of the cutting tool as the cutting tool contacts at least one registration position on the patient; and
generate, using the at least one registration position, the coordinate frame.

11. A method of controlling a robotic device, comprising:
capturing a plurality of positions of a cutting tool of the robotic device in a coordinate frame as the cutting tool contacts a plurality of locations on a patient;
generating, using the plurality of positions and a projection from the plurality of positions to a selected cut depth, a virtual boundary in the coordinate frame; and
controlling the robotic device using the virtual boundary to guide the cutting tool in executing a resection in accordance with the virtual boundary.

12. The method of claim 11, wherein the plurality of captured positions comprises three captured positions.

13. The method of claim 11, wherein generating the projection from the plurality of positions to the selected cut depth comprises:
fitting a plane to the plurality of positions;
determining a direction normal to the plane and into the patient; and
defining a plurality of vertices spaced apart from the plurality of positions in the direction by the selected cut depth,
wherein the virtual boundary intersects the plurality of vertices and the plurality of positions.

14. The method of claim 11, wherein generating the projection from the plurality of positions to the selected cut depth comprises projecting a cone from the plurality of positions such that the plurality of positions define a base of the cone and such that an apex of the cone is at the selected cut depth from the base.

15. The method of claim 11, wherein the virtual boundary is a hemisphere defined by the plurality of captured positions of the cutting tool, and wherein the virtual boundary is projected into the patient from the plurality of captured positions of the cutting tool.

16. The method of claim 11, wherein generating the virtual boundary in the coordinate frame is based on a surface geometry of a selected implant.

17. One or more non-transitory computer-readable media storing program instructions that, when executed by one or more processors, cause the one or more processors to perform operations comprising:
capturing a plurality of positions of a cutting tool of a robotic device in a coordinate frame as the cutting tool contacts a plurality of locations on a patient;
generating, using the plurality of positions and a projection from the plurality of positions to a selected cut depth, a virtual boundary in the coordinate frame; and
controlling the robotic device using the virtual boundary to guide the cutting tool in executing a resection in accordance with the virtual boundary.

18. The non-transitory computer-readable media of claim 17, wherein the plurality of captured positions comprises three captured positions.

19. The non-transitory computer-readable media of claim 17, wherein the plurality of captured positions are captured continuously as the cutting tool traces a portion of a joint of the patient.

20. The non-transitory computer-readable media of claim 17, wherein the virtual boundary is a hemisphere defined by the plurality of captured positions of the cutting tool, and wherein the virtual boundary is projected into the patient from the plurality of captured positions of the cutting tool.
